(19) United States
(12) Patent Application Publication
(10) Pub. No.: US 2011/0110886 A1
(43) Pub. Date: May 12, 2011

(54) SMALL MOLECULE INHIBITORS OF AUTOTAXIN AND METHODS OF USE

(75) Inventor: Demetrios Braddock, Guilford, CT (US)

(73) Assignee: Yale University

(21) Appl. No.: 12/993,397

(22) PCT Filed: Jun. 15, 2009
(86) PCT No.: PCT/US09/03565

§ 371 Date: Jan. 18, 2011

Related U.S. Application Data
(60) Provisional application No. 61/131,971, filed on Jun. 13, 2008.

(51) Int. Cl. A61K 31/675; C07F 9/6558; C07F 9/655; C07C 39/15; C07D 311/86; C07C 323/20; C07D 327/08; C07F 9/74; C07C 63/331; A61K 31/665; A61K 31/055; A61K 31/352; A61K 31/10; A61K 31/39; A61K 31/285; A61K 31/194; A61P 35/00; A61P 35/02; A61K 39/395; A61K 31/404; A61K 31/44; A61K 31/4439; A61K 31/506; A61K 38/20; A61K 38/46; A61K 33/24; A61K 38/21

(52) U.S. Cl. 424/85.2; 544/243; 549/220; 568/726; 549/392; 568/48; 549/16; 556/73; 562/488; 514/85; 514/100; 514/735; 514/454; 514/712; 514/434; 514/504; 514/568; 424/133.1; 514/414; 514/350; 514/338; 514/275; 424/94.6; 424/649; 424/85.7

(57) ABSTRACT

Autotaxin (ATX) is a prometastatic enzyme initially isolated from the conditioned media of human melanoma cells that stimulates a myriad of biological activities including angiogenesis and the promotion of cell growth, survival, and differentiation through the production of lysophosphatidic acid (LPA). ATX increases the aggressiveness and invasiveness of transformed cells, and ATX levels directly correlate with tumor stage and grade in several human malignancies. To study the role of ATX in the pathogenesis of malignant melanoma, we developed antibodies and small molecule inhibitors against recombinant human protein. Immunohistochemistry of paraffin embedded human tissue demonstrates that ATX levels are markedly increased in human primary and metastatic melanoma relative to benign nevi. Chemical screens identified several small molecule inhibitors with binding constants ranging from nanomolar to low micromolar. Cell migration and invasion assays with melanoma cell lines demonstrate that ATX markedly stimulates melanoma cell migration and invasion, an effect suppressed by ATX inhibitors. The migratory phenotype can be rescued by the addition of ATX's enzymatic product, LPA, confirming that the observed inhibition is linked to suppression of LPA production by ATX. Chemical analogues of the inhibitors demonstrate structure activity relationships important for ATX inhibition and indicate pathways for their optimization. These studies suggest that ATX is an approachable molecular target for the rational design of chemotherapeutic agents directed against human malignancies driven by the ATX/LPA axis, especially including malignant melanoma, among numerous others including breast and ovarian cancers.

### RELATED APPLICATIONS/CLAIM OF PRIORITY

This application claims the benefit of priority of U.S. provisional application Ser. No. 61/131,971, filed Jun. 13, 2008, entitled “Small Molecule Inhibitors of Autotaxin that Inhibit Cancer Cell Migration and Invasion”, the entire contents of which application is incorporated by reference herein.

### FIELD OF THE INVENTION

The present invention relates to compounds which have been discovered to inhibit growth and metastasis of cancer cells in patients with cancer. The compounds according to the invention may be used to treat cancer, to inhibit the metastatis, propagation, invasion and/or growth of cancer. The present invention is also directed to pharmaceutical compositions which comprise these compounds as well as methods for treating cancer.

### BACKGROUND OF THE INVENTION

Autotaxin (ATX) is a secreted glycoprotein member of the nucleotide pyrophosphatase/phosphodiesterase (NPP) family of enzymes that was first identified as a motility-stimulating factor in melanoma cells [1]. ATX has both the phosphodiesterase (PDE) activity expected of NPPs [2, 3] and also a lysophospholipase D (lysoPLD) activity, unique among this family [4-7]. Both PDE and lysoPLD activities occur at the same ATX active site, although PDE activity is considerably weaker and is unlikely to have physiological relevance [2, 4, 8].

ATX's lysoPLD activity generates lysophosphatidic acid (LPA) from lysophosphatidylcholine (LPC) [4, 5, 9], though it can hydrolyze other lysolipids as well, such as sphingosylphosphorylcholine (SPC) [9]. ATX is the sole source of extracellular LPA, as shown by transgenic animal experiments in which heterozygous ATX knockout mice possess half the ATX activity and serum LPA levels observed in their wildtype counterparts [10]. LPA mediates a broad range of biological activities through the activation of G-protein-coupled cell surface receptors to stimulate events central to organismal fate such as wound healing, brain development, and vascular remodeling [11].

While ATX is not responsible for oncogenic transformation, it has been demonstrated to increase tumor invasiveness, growth and metastasis, and neovascularization [12, 13]. In addition, recent studies of ATX knockout mice suggest that ATX contributes to tumor progression by stabilizing blood vessels in the vicinity of tumors [14]. The potent mitogenic activity of human ovarian cancer ascitic fluid is mediated by LPA and linked to ATX activity, and ATX is upregulated in tumor cells at the leading edge of the locally invasive human brain tumor glioblasoma multiforme [15]. ATX is increased in Hodgkins lymphoma (HD) cells which carry the Epstein-Barr virus (EBV), and is thought to mediate an aggressive phenotype in EBV positive HD [16]. In addition, LPA signaling plays a role in the motility and growth and metastasis of prostate cancer [17, 18], suggesting a role for ATX in prostatic adenocarcinoma.

The increased expression of ATX in a wide variety of human tumors relative to normal tissues has been established by multiple complementary techniques including the quantification of mRNA levels by in situ hybridization and quantitative PCR, and the quantification of protein levels by immunohistochemistry and western blotting. ATX was first cultured from the conditioned media of human melanoma cells [1], and three out of four (75%) melanoma cell lines tested were reported to over express and secrete ATX (including cell line A2058) [19]. Other tumors with increased ATX expression include breast cancer—where it correlates with tumor invasiveness [20], teratocarcinoma [3], neuroblastoma—where it correlates with the more aggressive and lethal variant commonly observed in older patients [21], glioblastoma—where expression is greater in the leading edge of invasive tumor cells as compared to the tumor core [15, 22], lung carcinoma—where over expression is found in seven out of twelve (58%) tumor cell lines [23], thyroid carcinoma—where it correlates with the aggressive anaplastic variant of thyroid carcinoma compared with the less aggressive follicular thyroid carcinoma cell lines [24], and ovarian cancer—where astronomical levels of the enzymatic product of ATX are found in the malignant ascitic fluids [25-28]. Taken together, the data indicates that ATX functions as a tumor motility and angiogenic factor, stimulating multiple facets of the metastatic cascade to promote aggressive variants of human malignancies.

Primary malignant melanoma usually presents with cutaneous lesions that can be readily treated surgically, but metastatic melanoma is poorly controlled surgically and chemotherapeutically and often follows an ominous clinical course. ATX is an extracellular prometastatic enzyme and therefore an attractive molecular target for melanoma since inhibitory compounds can reach the target site without having to cross the cell membrane. LPA analogues are effective ATX inhibitors [29-32], and successfully inhibit tumor growth in animal models [33], but LPA mimics could also bind and activate LPA receptors initiating the signaling cascades an ATX inhibitor is intended to stop. Two recent studies identified several small molecule ATX inhibitors [32, 33]. However, the binding constants of the inhibitors were not measured, nor were the effects on cancer cell migration.

### BRIEF DESCRIPTION OF THE FIGURES

FIG. 1. A-D shows the steady-state ATX-dependent hydrolysis of pNP-TMP and FS-3. A. Time courses of pNP-TMP hydrolysis by 75 nM ATX assayed by absorbance at 405 nm. The curves represent (lower to upper) 0, 0.1, 0.6, 0.9, 1.5, 3, 7, 15, 20, 30 and 40 mM pNP-TMP. B. [pNP-TMP]-dependence of hydrolysis rate. The solid line represents the best fit to a rectangular hyperbola (Eq. 1). The K_{M} for pNP-TMP is 1.4 mM and the k_{cat} for hydrolysis is 1.6 s⁻¹. C. Time courses of FS-3 hydrolysis by 200 nM ATX assayed by fluorescence (λₑₓ=485 nm, λₑₘ=520 nm). The curves represent (lower to upper): 0, 1, 2, 4, 24, and 36 μM FS-3. D. [FS-3]-dependence of hydrolysis rate. The solid line represents the best fit to a rectangular hyperbola. The K_{M} for FS-3 is 4.5 μM the k_{cat} was normalized to compare multiple days. Error bars represent standard deviation (n=4).

FIG. 2 shows the structures of substrates, products, and certain lead inhibitors. FIG. 2A shows the hydrolysis of pNP-TMP B. FIG. 2B shows the hydrolysis of FS-3. C. Structure of hexachlorophene, merbromin. Bithionol and their analogs -2,2′-Methylenebis(4-chlorophenol), Eosin Y and RJC 03297, respectively. D. Structure of NSC 48300 and its analogs. The percentage inhibition of fluorescent product when compared to ATX alone in the absence of inhibitor is displayed below each compound. The assay conditions are 300 nM ATX in the presence or absence of 10 μM compound over 30 minutes.

FIG. 3 shows the Inhibitor effects on ATX lysoPLD activity. A. Time courses of FS-3 hydrolysis in the presence of NSC 48300 at 2 μM FS-3. Top to bottom: 0, 0.5, 1 μM NSC 48300. B. [Hexachlorophene]-dependence of ATX activity. Top to bottom: 0, 200, and 300 μM hexachlorophene. The solid lines represent the best fits of averaged data points to rectangular hyperbolas and the error bars represent standard deviation (n=4). The data has been corrected for internal filtering. C. [Bithionol]-dependence of ATX activity. Top to bottom: 0, 150 and 200 μM. Solid lines are the best fits of averaged data points (n=2) to rectangular hyperbolas, and the error bars represent standard error. D. [NSC 48300]-dependence of ATX activity. Top to bottom: 0, 0.5, 1 μM. Solid lines represent the best fits of averaged data points (n=2) to rectangular hyperbolas, and the error bars represent the standard deviation.

FIG. 4 shows the effect of inhibitors on melanoma cell migration. A. Cell migration of melanoma cells 4 hours after the addition of (from left to right) 28 μL media, 75 nM LPA, or 50 nM ATX. B. Average of nine melanoma cell motility assays with media, media and 0.1% DMSO, 75 nm LPA, or 50 nM ATX ±inhibitors in the absence and presence of LPA. Error bars represent one standard deviation. The addition of LPA to the experimental groups containing inhibitors (LPA rescue) completely abrogated the small molecule inhibitory effect on cell motility (see discussion). The LPA rescued motility was significantly increased over the inhibited motility for all inhibitors (p≦0.001). The increase in motility with both LPA and ATX was statistically significant (p≦0.0001). C. Average melanoma cell motility as a function of inhibitor concentration. Left—inhibitors merbromin (black), bithionol (purple), hexachlorophene (red), and NSC48300 (blue). The solid line represents the best fit to a rectangular hyperbola, which was only modeled for NSC 48300, as the other inhibitors could not be fit to a single binding event (see discussion). Right-Average melanoma cell motility as a function of blebbistatin concentration in the presence of 50 nM ATX (black) or 75 nM LPA (purple). The solid line represents the best fit to a rectangular hyperbola, modeled to a single binding event.

FIG. 5 shows the effect of inhibitors on melanoma, ovarian cancer, and breast cancer invasion. Average of three cell invasion assays showing melanoma cell invasion (Panel A), primary ovarian cancer cell invasion (Panel B), or breast cancer cell invasion (Panel C) in the presence of media, 0.1% DMSO, 75 nM LPA, and 50 nM ATX ±the indicated concentrations of NSC48300 or bithionol in the absence and presence of LPA. Error bars represent one standard deviation. The addition of LPA to the experimental groups containing inhibitors (LPA rescue) completely abrogated the small molecule inhibitory effect on cell motility (see discussion) with the exception of 5 μM concentration of NSC48300 on A2058 cells. The LPA rescued motility in the other experimental groups was significantly increased over the inhibited motility for all inhibitors (p≦0.001). The increase in motility with both LPA and ATX was statistically significant (p≦0.0001).

FIG. 6, Table 1, shows a summary of ATX steady-state parameters.

FIG. 7 shows the expression of ATX in human tissue microarrays. Tissue microarrays of normal skin, benign nevi, primary melanoma, and metastatic melanoma (10 examples of each) were stained with chicken polyclonal antibody raised against recombinant ATX. All images at 60×. A. Normal skin showing adnexal structures B. Benign nevi C. Primary Melanoma D. Metastatic Melanoma

FIG. 8. A-D shows the inhibitor effects on ATX PDE activity. A. [Hexachlorophene]-dependence of ATX activity. Top to bottom: 0, 1, 10, and 100 μM hexachlorophene. The solid lines represent the best fits to rectangular hyperbolas. B. [Merbromin]-dependence of ATX activity. Top to bottom: 0, 1, 10 and 100 μM. Solid lines are the best fits to rectangular hyperbolas. C. [Bithionol]-dependence of ATX activity. Solid lines are the best fits to rectangular hyperbolas. D. [NSC 48300]-dependence of ATX activity. Top to bottom: 0, 1, and 2 μM NSC 48300. Top curve represents the average ATX activity.

FIG. 9A-B show the effect of LPA on cell migration in a panel of melanoma cell lines.

9A. Shows the cell migration of a panel of 5 melanoma cells lines 3 hours after the addition of media (white bars) or 75 nM LPA (black bars). Melanoma cells, MeI 28, MeI 888, and YUSAC2, did not migrate following a 3-hour incubation with 75 nM LPA, while A2058 and HTB 63 responded to LPA. All error bars in this figure indicate the standard error of the mean for nine replicates. The increase in cell motility with LPA was statistically significant (α</=0.001).

9B. Shows the effect of bithionol on cell migration of melanoma cell line HTB 63. Cell migration of HTB 63 cells 3 hours after the addition of (from left to right) media, 75 nM LPA, or 50 nM ATX ±the indicated concentrations of bithionol, or bithionol and 75 nM LPA. All error bars in this figure indicate the standard error of the mean for nine replicates. The addition of LPA to the experimental groups containing inhibitors (LPA rescue) completely abrogated the small molecule inhibitory effect on cell motility (see discussion). The LPA rescued motility was significantly increased over the inhibited motility for all inhibitors (p≦0.001). The increased motility with both LPA and ATX was statistically significant (p≦0.0001).

FIGS. 10 A-C show the effects of inhibitor analogs on ATX PDE activity. A. [2,2′-Methylenebis(4-chlorophenol)]-dependence of ATX. Top to bottom: 0, 400 and 600 μM 2,2′-Methylenebis(4-chlorophenol). Solid lines are best fits to rectangular hyperbolas. B. [Eosin Y]-dependence of ATX activity. Top to bottom: 0, 40 and 50 μM Eosin Y. Solid lines are best fits to rectangular hyperbolas. C. [RJC 03297]-dependence of ATX activity with 1.5 mM pNP-TMP.

FIGS. 11 A-C show the effects of inhibitor analogs on ATX lyso-PLD activity. A. [2,2′-Methylenebis(4-chlorophenol)]-dependence of ATX. Top to bottom: 0, 400 and 600 μM 2,2′-Methylenebis(4-chlorophenol). Solid lines are best fits to rectangular hyperbolas. B. [Eosin Y]-dependence of ATX activity. Top to bottom: 0, 40 and 50 μM Eosin Y. Solid lines are best fits to rectangular hyperbolas. C. [RJC 03297]-dependence of ATX activity with 1.5 mM pNP-TMP.

FIGS. 12 and 13 show that ATX stimulates pericyte migration, which is a key process in the formation of mature and stable angiogenesis. These are cell migration assays in which pericytes are plated on one side of a membrane and either ATX or LPA is added to the other side, and the migration of the cells across a membrane with 8 uM pores is quantified.

FIG. 14—shows the effects of bithionol suppression of melanoma invasion and metastasis in vivo. Nude mice were inoculated with human melanoma cell line A2058 subcutaneously in the right flank and observed until the tumor was palpable (about 5-8 days). The mice were then treated with oral gavage of a bithionol-cyclodextrin suspension (A-D) or a cyclodextrin suspension alone (E-H). Panels A-D.—All 5 mice treated with bithionol lacked gross evidence of metastatic melanoma upon necropsy. Dissection of the tumor from the underlying fascia was easily accomplished for all animals (demonstrated in A&B). Microscopically, the tumors appeared as self-contained balls of tumor cells (C) with a pushing border. Higher powered examination of the tumor border reveals tumor encapsulated by benign fibroblasts and connective tissue (D). Panels E-H—3 of the 4 untreated mice showed gross and/or microscopic evidence of invasive melanoma. Two of the untreated mice developed large tumors in the right hindquarter (cyan arrow, E) that grossly invaded the right gluteous maximus and could not be dissected without amputation (E&F). Microscopic examination of the tumor revealed an invasive tumor which encompassed the muscle and bone of the right leg (G). Higher power examination of the tumor interface reveals tumor cells invading into and splaying apart normal muscle cells (H). Of the remaining untreated mice, one developed a modest sized tumor with microscopic evidence of invasion, and a one developed a tumor without gross or microscopic evidence of invasive tumor.

FIG. 15: Effect of ATX inhibition on breast cancer tumor growth. Murine mouse carcinoma cell line 66cl4 was injected into the left mammary fat pad and allowed to grow for 48 hours. The mice were then orally dosed with alternate daily doses of bithionol-cyclodextrin (6 animals, 60/mg/kg) or cyclodextrin alone (6 animals) for 24 days. A. Scatterplot of the weights of tumor for both experimental groups. B. Average tumor weights of both experimental groups. The average tumor weight of the control animals was 0.297±0.116 g, and the average weight of the dosed animals was 0.141±0.107 g. the difference in weight was statistically significant (p=0.019). C. The weights of the animals in the two experimental groups did not differ significantly from each other over the course of the experiment.

### BRIEF DESCRIPTION AND OBJECTS OF THE INVENTION

The present invention relates to the establishment of autotaxin ATX as a molecular target to treat cancer and inhibit growth and metastasis of a variety of cancers, in particular, malignant melanoma growth and metastasis by verifying over-expression in primary and metastatic melanoma, identifying small molecule ATX inhibitors, and quantifying their effects on melanoma cell motility and invasion.

The present invention relates to compounds according to the chemical structure:

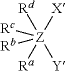

Where Z is a 5- or 6-membered ring containing up to four heteroatoms (O, S, N) or together with X′ or Y′, forms an optionally substituted fused ring system containing two or three rings, wherein said rings may be saturated or unsaturated, carbocyclic or heterocyclic (including aromatic or heteroaromatic);
X′ and Y′ are each independently H, optionally substituted heterocyclic, aryl or heteroaryl, wherein said heterocyclic, aryl or heteroaryl is optionally bonded to said Z group through a linker group L, halogen, an optionally substituted alkyl, OR′, where R′ is H, an optionally substituted C₁-C₆ alkyl (preferably C₁-C₃ alkyl), —C(O)—(C₁-C₆ alkyl),
—C(O)R″, where R″ is H, OH, an optionally substituted C₁-C₆ alkyl, O—(C₁-C₆ alkyl), NR^{Na}R^{Nb}, where R^{Na} is H or a C₁-C₆ alkyl and R^{Nb} is H, an optionally substituted C₁-C₃ alkyl or a C(O)R^{Nc} or C(O)OR^{Nc} group, where R^{Nc} is an optionally substituted C₁-C₁₂ hydrocarbyl group (including an aryl group), an optionally substituted saturated or unsaturated heterocyclic group (including a heteroaromatic group), a —AsO₃ group, a
Hg—O—R^{HG} group where R^{HG} is H or a C₁-C₃ alkyl group, a S(O)ₖR^{f} group, where R^{f} is H, an optionally substituted C₁-C₁₂ hydrocarbyl, heterocyclic or heteroaromatic group or a NR^{Nd}R^{Ne} group, where R^{Nd} is H or an optionally substituted C₁-C₆ hydrocarbyl group and R^{Ne} is H, or an optionally substituted C₁-C₁₂ hydrocarbyl group (preferably substituted with a S(O)ₖR^{fa} group, where R^{fa} is H, an optionally substituted C₁-C₁₂ hydrocarbyl, heterocyclic or heteroaromatic group, or a NR^{Nfa}R^{Nfe} group, where R^{Nfa} is H or an optionally substituted C₁-C₆ hydrocarbyl group and R^{Nfe} is H or an optionally substituted C₁-C₁₂ hydrocarbyl group;
L is a linker group of the general structure:

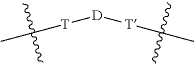

Where T and T′ are each independently a bond, —(CH₂)ᵢ—O, —(CH₂)ᵢ—S, —(CH₂); —N—R,

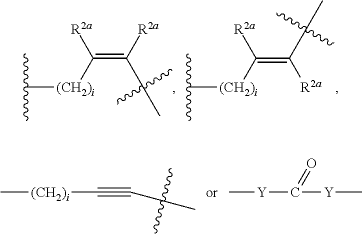

wherein said —(CH₂); group, if present in T or T′, is bonded to Z or X or Y;
R is H, or a C₁-C₃ alkyl group;
R^{2a} is H or a C₁-C₃ alkyl group;
Each Y is independently a bond, O, S or N—R;
Each i is independently 0, 1, 2 or 3;
k is 0, 1 or 2;

### D is O, S, or N—H;

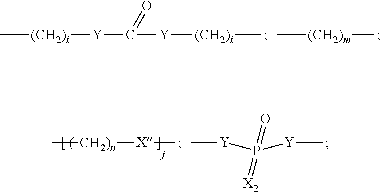

Where X₂ is O or is absent (along with the double bond);
i is the same as described above;
j is 1, 2, 3 or 4,
m is 1, 2, 3, 4, 5 or 6;
n is 1, 2 or 3; and

### X″ is O, S or N—R;

R is H, or a C₁-C₃ alkyl group;
R^{a}, R^{b}, R^{c} and R^{d} are each independently absent (because the heteroatom is O or S and cannot accommodate a substituent) H, halogen (preferably F, Cl or Br), optionally substituted C₁-C₆ alkyl, OH, CN, NO₂, C(O)H, C(O)OH, O—(C₁-C₆ alkyl), C(O)(C₁-C₆ alkyl), C(O)—O—(C₁-C₆ alkyl), —O—C(O)—(C₁-C₆ alkyl), a AsO₃ group or a Hg—O—R^{HG} group where R^{HG} is H or a C₁-C₃ alkyl group, with the proviso that no more than two substituents contain Hg or As;
or a pharmaceutically acceptable salt, solvate or polymorph thereof.

In certain aspects of the invention, compounds according to the present invention exhibit symmetry of the entire molecule or of substantial portions of the molecule, e.g., a tricyclic moiety, an aryl moiety or other moiety of the compound.

In certain aspects of the invention, preferred compounds are biphenyl compounds or bridged biphenyl compounds according to the chemical structure:

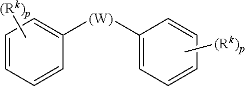

Where W is (CH₂)ᵢ, O, S or NR_{T};
i is 0, 1, 2 or 3;
R_{T} is H or C₁-C₃ alkyl (preferably H or CH₃);
Each R^{k} is independently OH, halogen (F, Cl, Br, I), C(O)H, C(O)OH, O—(C₁-C₆ alkyl), C(O)(C₁-C₆ alkyl), C(O)—O—(C₁-Cₒ alkyl), —O—C(O)—(C₁-C₆ alkyl), a AsO₃ group or a Hg—O—R^{HG} group where R^{HG} is H or a C₁-C₃ alkyl group, with the proviso that no more than two substituents contain Hg or As
p is 1, 2, 3, 4 or 5, preferably 1-3;
or a pharmaceutical salt solvate or polymorph thereof.

In certain preferred aspects of the invention, the above compound is symmetrical, i.e., the aryl groups on either side of the (W) linker portion of the molecule contain substituents such that the entire molecule is symmetrical-contains the identical substituents on both sides of the molecule in a manner such there is symmetric about a y-axis which runs through the V group of tricyclic moiety.

In other aspects of the invention, preferred compounds are tricyclic compounds according to the chemical structure:

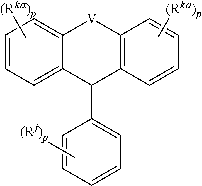

### Where V is O, S or NR_{T};

R_{T} is H or C₁-C₃ alkyl (preferably H or CH₃);
Each R^{ka} is independently OH, CN, NO₂, halogen (F, Cl, Br, I), C(O)H, C(O)OH, O—(C₁-C₆ alkyl), C(O)(C₁-C₆ alkyl), C(O)—O—(C₁-C₆ alkyl), —O—C(O)—(C₁-C₆ alkyl), a AsO₃ group or a Hg—O—R^{HG} group where R^{HG} is H or a C₁-C₃ alkyl group, with the proviso that no more than two substituents R^{ka} contain Hg or As;
Each R^{j} is independently OH, halogen (F, Cl, Br, I), C(O)H, C(O)OH, O—(C₁-C₆ alkyl), C(O)(C₁-C₆ alkyl), C(O)—O—(C₁-C₆ alkyl), —O—C(O)—(C₁-C₆ alkyl), a AsO₃ group or a Hg—O—R^{HG} group where R^{HG} is H or a C₁-C₃ alkyl group, with the proviso that no more than two substituents R^{j} contain Hg or As;
p is 1, 2, 3, 4 or 5, preferably 1-3;
or a pharmaceutical salt, solvate or polymorph thereof.

In certain preferred aspects of the invention, the tricyclic moiety of the above compound is symmetrical, i.e., the tricyclic portion of the molecule contains substituents such that the tricyclic moiety is symmetrical-contains the identical substituents on both sides of the molecule in a manner such there is symmetric about a y-axis which runs through the V group of tricyclic moiety.

In other aspects of the invention, preferred compounds are saturated or unsaturated 5-membered heterocyclic rings according to the formula:

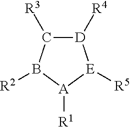

Where A, B, C, D or E are each a carbon, nitrogen, oxygen or sulphur atom (preferably carbon or nitrogen) with the proviso that at least two of A, B, C, D and E are carbon (preferably at least three of A, B, C, D and E are carbon and preferably A, C and E are carbon atoms and the other two atoms are nitrogen atoms);
R¹, R² and R³ K are each independently absent (because the heteroatom cannot accommodate a substituent), H, halogen, an optionally substituted C₁-C₆ alkyl group, OH, O—(C₁-C₆ alkyl), O—C(O)—(C₁-C₆ alkyl), C(O)—(C₁-C₆ alkyl), C(O)—O—(C₁-C₆ alkyl), a AsO₃ group or a Hg—O—R^{HG} group where R^{HG} is H or a C₁-C₃ alkyl group, with the proviso that no more than two substituents of R¹, R² and R³ contain Hg or As or an optionally substituted 5- or 6 membered saturated or unsaturated carbocyclic or heterocyclic ring;
R⁴ and R⁵ are each independently absent (because the heteroatom cannot accommodate a substituent), H, halogen, optionally substituted C₁-C₆ alkyl or a NR^{g}R^{h}, where R^{g} is H, a C₁-C₃ group and R^{h} is H, a C₁-C₆ hydrocarbyl group or a C(O)—R^{h′} group where R^{h′} is an optionally substituted C₁-C₁₂ hydrocarbyl group or an optionally substituted heterocyclic group, or together R⁴ and R⁵ together form an optionally substituted five or six-membered saturated or unsaturated carbocyclic or heterocyclic group; or
a pharmaceutically acceptable salt, solvate or polymorph thereof.

In certain aspects of the invention, the compounds which are described above are symmetrical in presentation, i.e., substituents on either side of a moiety are identical (present a mirror image) when a y-axis is drawn through the middle of the symmetrical molecule or moiety.

Alternative preferred embodiments according to the present invention relate to compounds according to the structure:

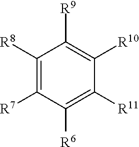

Where R⁶, R⁷, R⁸ and R⁹ are each independently selected from H, halogen, optionally substituted C₁-C₆ alkyl, OH, O—(C₁-C₆ alkyl), O—C(O)—(C₁-C₆ alkyl), C(O)—(C₁-C₆ alkyl), and C(O)—O—(C₁-C₆ alkyl), a AsO₃ group or a Hg—O—R^{HG} group where R^{HG} is H or a C₁-C₃ alkyl group or together R⁸ and R⁹ form an optionally substituted 5- or 6-membered saturated or unsaturated carbocyclic or heterocyclic ring (preferably optionally substituted aromatic or heteroaromatic);
R¹⁰ is a halogen, optionally substituted C₁-C₆ alkyl, OH, O—(C₁-C₆ alkyl), O—C(O)—(C₁-C₆ alkyl), C(O)—(C₁-C₆ alkyl), and C(O)—O—(C₁-C₆ alkyl), a AsO₃ group or a Hg—O—R^{HG} group where R^{HG} is H or a C₁-C₃ alkyl group,

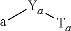

group, where Yₐ is S, an optionally substituted (CH₂)_{q} group where q is 0, 1, 2, 3 or 4 (preferably 1) or an amine group which is optionally substituted with a single C₁-C₃ alkyl group and Tₐ is an optionally substituted aromatic or heteroaromatic group, or a S(O)ₖR^{f} group, where k is 0, 1 or 2 and R^{f} is H, an optionally substituted C₁-C₁₂ hydrocarbyl, heterocyclic or heteroaromatic group or a NR^{Nd}R^{Ne} group, where R^{Nd} is H or an optionally substituted C₁-C₆ hydrocarbyl group and R^{N′} is H, or an optionally substituted C₁-C₁₂ hydrocarbyl group (preferably substituted with a S(O)ₖR^{fa} group, where R^{fa} is H, an optionally substituted C₁-C₁₂ hydrocarbyl, heterocyclic or heteroaromatic group, or a NR^{Nfa}R^{Nfe} group, where R^{Nfa} is H or an optionally substituted C₁-C₆ hydrocarbyl group and R^{Nfe} is H or an optionally substituted C₁-C₁₂ hydrocarbyl group, or together with R¹¹ and the aromatic ring to which they are attached, form an optionally substituted saturated or unsaturated carbocyclic or heterocyclic tricyclic ring system; and R¹¹ is H, halogen, optionally substituted C₁-C₆ alkyl, OH, O—(C₁-C₆ alkyl), O—C(O)—(C₁-C₆ alkyl), C(O)—(C₁-C₆ alkyl), and C(O)—O—(C₁-C₆ alkyl), a AsO₃ group or a Hg—O—R^{HG} group where R^{HG} is H or a C₁-C₃ alkyl group an optionally substituted C₁-C₁₂ hydrocarbyl group, an optionally substituted heterocyclic group (preferably, an optionally substituted heteroaryl group) or together with R¹⁰ and the aromatic ring to which they are attached, form an optionally substituted saturated or unsaturated carbocyclic or heterocyclic tricyclic ring system; or
a pharmaceutically acceptable salt, solvate and polymorph thereof.

In certain aspects of the invention, the compounds which are described above are symmetrical in presentation, i.e., substituents on either side of an aryl moiety are identical (present a mirror image) when a y-axis is drawn through the middle of the symmetrical molecule or moiety.

Pharmaceutical compositions according to the present invention comprise a compound according to the present invention in an effective amount to treat cancer by inhibiting the growth and metastasis, propagation, invasion and/or growth of cancer in a patient, optionally (and preferably) in combination with a carrier, additive or excipient. Additional pharmaceutical compositions comprise an effective amount of a compound as otherwise described hereinabove, in combination with an additional anticancer or other bioactive agent, optionally in combination with a pharmaceutically acceptable carrier, additive or excipient.

A method for the treatment of cancer according to the present invention comprises administering an effective amount of a compound as otherwise described hereinabove, optionally in combination with a pharmaceutically acceptable carrier, additive or excipient to a patient in need of therapy. Additional anticancer agents, as well as bioactive agents may be employed in this method as well. The method may be used to treat cancer, including cancerous tumors directly by inhibiting antiogenesis. Methods of the present invention may also be used to inhibit, reduce the likelihood or prevent growth and metastasis in patients having cancer, especially including for example, brain cancer (gliablastoma), skin cancer (melanoma), breast, prostate, ovarian, lung, stomach, colon and thyroid cancers, among others.

In an alternative embodiment, a method for inhibiting autotoxin comprises exposing autotaxin to an effective amount of a compound as otherwise described hereinabove. The method of inhibiting autotaxin can be used in an assay to test for the potential anticancer/antimetastatic activity of a compound or, alternatively, in a patient to be treated, such as a cancer patient.

A method of inhibiting angiogenesis in a patient represents another aspect of the present invention. In this method, one or more compounds according to the invention may be administered to a patient in an effective amount to inhibit angiogenesis in a patient or treat an angiogenic (angiogenesis related) disease, including, among others, macular degeneration, especially including exudative (wet) macular degeneration and diabetic retinopathy.

### DETAILED DESCRIPTION OF THE INVENTION

The present invention is directed to a number of inhibitors of ATX with inhibitory constants in the therapeutic range, including nanomolar or low micromolar range. The inhibitors have been further validated using in vitro cell motility (Boyden Chamber) and cell invasion (matrigel) assays on human melanoma cell lines. Structural analogs of a subset of the inhibitors reveal chemical moieties responsible for inhibition. A chemical scaffold with high affinity (nM) to ATX has been identified as well as numerous analogs of same, as well as inhibitors with low in vivo toxicity, including agents which are orally bio-available and in one instance, has undergone FDA approval.

The present invention provides a large number of anti-metastatic compounds which are useful for treating cancer to prevent its growth, metastasis, invasion and/or propagation, in elucidating the pathogenesis of a number of cancers, including melanoma and their spread and growth and metastasis, and may point to the rational design of future chemotherapeutic agents in the treatment of a number of cancers, including melanoma.

Pharmaceutical compositions according to the present invention comprise an effective amount of at least one compound as described hereinabove, optionally (preferably) in combination with a pharmaceutically acceptable carrier, additive or excipient. Additional pharmaceutical compositions comprise effective amounts of compounds according to the present invention in combination with at least one additional anti-cancer agent or bioactive agent, in combination with a pharmaceutically acceptable carrier, additive or excipient.

In describing the present invention, the following terms shall be used. In instances where a term is not specifically defined herein, that term is given its typical meaning by those of ordinary skill when using that term within the context of its use.

The term “patient” or “subject” is used throughout the specification to describe an animal, generally a mammal and preferably a human, to whom treatment, including prophylactic treatment, with the compositions according to the present invention is provided. For treatment of those infections, conditions or disease states which are specific for a specific animal such as a human patient, the term patient refers to that specific animal.

The term “compound”, as used herein, unless otherwise indicated, refers to any specific chemical compound disclosed herein. Within its use in context, the term generally refers to a single compound or its various racemic, enantiomerically enriched (to at least 75%, 85%, 95%, 98%, 99% or 99+% enantiomeric enrichment or various prodrug or derivative forms as otherwise described herein, including pharmaceutically acceptable salts solvates, polymorphs or enantiomers thereof. Preferred compounds according to the present invention exhibit little, if any toxicity, to host cells in treating cancer or other disease state or condition.

The term “effective” is used herein, unless otherwise indicated, to describe an amount of a compound which, in context, is used to produce or effect an intended result, whether that result relates to the treatment of a cancer, and in particular, inhibition of growth and metastasis of a cancer or cancers or the treatment of an angiogenic disease state or condition, in particular, macular degeneration, especially exudative (wet) macular degeneration or diabetic retinopathy. In certain aspects related to the coadministration of a compound according to the present invention with another anticancer agent, the present invention relates to the enhancement of the anti-cancer effect of said other anti-cancer compound. This term subsumes all other effective amount or effective concentration terms which are otherwise described in the present application. With respect to an anti-cancer effect, that effect may be one or more of inhibiting further growth of tumor or cancer cells, reducing the likelihood or eliminating growth and metastasis or producing cell death in the tumor or cancer cells, resulting in a shrinkage of the tumor or a reduction in the number of cancer cells or preventing the regrowth of a tumor or cancer after the patient's tumor or cancer is in remission. As indicated, compounds according to the present invention or their derivatives may exhibit an anti-cancer effect alone (principally, by inhibiting metastastis) and/or may enhance the ability of another anti-cancer agent to exhibit an anti-cancer effect in an additive or synergistic manner (i.e., more than additive). In aspects relating to the treatment or resolution of an angiogenic disorder, an effective amount of a compound according to the present invention may be used to eliminate, inhibit, resolve or ameliorate the angiogenic disease state or condition or conditions secondary to an angiogenic disease state or condition.

The term “independently” is used herein to indicate that the variable, which is independently applied, varies independently from application to application.

The term “symmetrical” is used herein to describe a molecule or a moiety within a molecule which is symmetrical, i.e. the molecule or moiety contains substituents such that the identical substituents are presented in a mirror-image fashion when a y-axis (or x-axis, depending on presentation) is drawn through the molecule. In certain aspects of the invention, compounds or significant moieties within the compounds are symmetrical.

The term “hydrocarbyl” shall mean within its use in context, a radical containing carbon and hydrogen atoms, preferably containing between 1 and 12 carbon atoms. Such term may also include cyclic groups and unsaturated groups such as aromatic groups, within context. A substituted hydrocarbyl group is a hydrocarbyl group where at least one hydrogen atom is substituted by another moiety, as described below. The term “alkyl” shall mean within its use in context a fully saturated C₁-C₁₂ hydrocarbon linear, branch-chained or cyclic radical, preferably a C₁-C₄, even more preferably a C₁-C₃ linear, branch-chained or cyclic fully saturated hydrocarbon radical. The term “alkenyl” is used to describe a hydrocarbon group containing at least two carbon atoms, similar to an alkyl group which contains at least one carbon-carbon double bond. The term “alkynyl” is used to describe a hydrocarbon group containing at least two carbon atoms similar to an alkyl group which contains at least one carbon-carbon triple bond. Unsaturated hydrocarbyl groups are anticipated for use in the present invention. The terms “alkylene” and “alkenylene” may be used to describe alkyl and alkenyl divalent radicals generally of up to 12 carbon units in length and preferably no greater than about 6 carbon units per length (for example, 1-3 carbon units in length) and may be subsumed under the terms alkyl and alkenyl, especially when referring to substituents or substituted.

The term “aromatic” or “aryl” shall mean within its context a substituted or unsubstituted monovalent carbocyclic aromatic radical having a single ring (e.g., phenyl) or multiple condensed rings (e.g., naphthyl, anthracene, phenanthrene). Other examples include optionally substituted heterocyclic aromatic ring groups (“heteroaromatic” or “heteroaryl”) having one or more nitrogen, oxygen, or sulfur atoms in the ring, such as imidazolyl, furyl, pyrrolyl, pyridyl, thiophene, thiazole, indolyl, quinoline, pyridine, pyridone, pyrimidine, indole, quinoline, isoquinoline, quinolizine, phthalazine, naphthyridine, quinazoline, cinnoline, acridine, benzothiophene, benzofuran, thiazole, benzothiazole, phenothiazine, indole, quinoline, isoquinoline, quinolizine, phthalazine, naphthyridine, quinazoline, cinnoline, acridine, benzothiophene, benzofuran, benzothiazole, pyrrolopyrimidine, pyrrolopyrazine, furopyrimidine and phenothiazineamong numerous others. The preferred aryl group in compounds according to the present invention is a phenyl or a substituted phenyl group. Aromatic groups according to the invention may be a single ring, bicyclic or tricyclic. It is noted that heteroaromatic or heteroaryl compounds, depending upon the use of the term in context, are also generally subsumed under the general term “heterocycle”.

The term “heterocycle” shall mean an optionally substituted moiety which is cyclic and contains at least one atom other than a carbon atom, such as a nitrogen, sulfur, oxygen or other atom. A heterocycle according to the present invention is an optionally substituted imidazole, a piperazine (including piperazinone), piperidine, furan, pyrrole, imidazole, thiazole, oxazole or isoxazole group, among others, including bicyclic and tricyclic ring systems, as set forth below. Depending upon its use in context, a heterocyclic ring may be saturated and/or unsaturated and therefore subsumes the term heteroaryl or heteroaromatic. The term “heterocyclic group” as used herein refers to an aromatic or non-aromatic cyclic group having 3 to 14 atoms forming the cyclic ring(s) and including at least one hetero atom such as nitrogen, sulfur or oxygen among the atoms forming the cyclic ring, which is a “3- to 14-membered aromatic heterocyclic group” (also, “heteroaryl” or “heteroaromatic”) or a “3- to 14-membered non-aromatic heterocyclic group” or heterocyclic group. Heterocyclic groups according to the invention may be a single ring, bicyclic or tricyclic. Exemplary heterocyclic groups include aziridine, pyrrole, pyridine, pyridone, pyridazine, pyrimidine, pyrazine, pyrazole, imidazole, triazole, tetrazole, indole, isoindole, indolizine, purine, indazole, quinoline, isoquinoline, quinolizine, phthalazine, naphthyridine, quinoxaline, quinazoline, cinnoline, pteridine, imidazopyridine, imidazotriazine, pyrazinopyridazine, acridine, phenanthridine, carbazole, carbazoline, perimidine, phenanthroline, phenacene, oxadiazole, benzimidazole, pyrrolopyridine, pyrrolopyrimidine and pyridopyrimidine; sulfur-containing aromatic heterocycles such as thiophene and benzothiophene; oxygen-containing aromatic heterocycles such as furan, pyran, cyclopentapyran, benzofuran and isobenzofuran; and aromatic heterocycles comprising 2 or more hetero atoms selected from among nitrogen, sulfur and oxygen, such as thiazole, thiadizole, isothiazole, benzoxazole, benzothiazole, benzothiadiazole, phenothiazine, isoxazole, furazan, phenoxazine, pyrazoloxazole, imidazothiazole, thienofuran, furopyrrole, pyridoxazine, furopyridine, furopyrimidine, thienopyrimidine and oxazole. As examples of the “3- to 14-membered aromatic heterocyclic group” there may be mentioned preferably, pyridine, triazine, pyridone, pyrimidine, imidazole, indole, quinoline, isoquinoline, quinolizine, phthalazine, naphthyridine, quinazoline, cinnoline, acridine, phenacene, thiophene, benzothiophene, furan, pyran, benzofuran, thiazole, benzthiazole, phenothiazine, pyrrolopyrimidine, furopyridine and thienopyrimidine, more preferably pyridine, thiophene, benzothiophene, thiazole, benzothiazole, quinoline, quinazoline, cinnoline, pyrrolopyrimidine, pyrimidine, furopyridine and thienopyrimidine

The term “unsubstituted” shall mean substituted only with hydrogen atoms. The term “substituted” shall mean, within the chemical context of the compound defined, a substituent (each of which substituent may itself be substituted) selected from a hydrocarbyl (which may be substituted itself, preferably with an optionally substituted alkyl or halogen group, particularly a bromo or fluoro group, among others), preferably an alkyl (generally, no greater than about 12 carbon units in length), an optionally substituted aryl (which also may be heteroaryl and may include an alkylenearyl or alkyleneheteroaryl), an optionally substituted heterocycle (especially including an alkyleneheterocycle), CF₃, halogen, thiol, hydroxyl, carboxyl, oxygen (to form a keto group), C₁-C₈ alkoxy, CN, nitro, an optionally substituted amine (e.g. an alkyleneamine or a C₁-C₆ monoalkyl or dialkyl amine), C₁-C₈ acyl, C₁-C₈ alkylester, C₁-C₈ alkyleneacyl (keto), C₁-C₈ alkylene ester, carboxylic acid, alkylene carboxylic acid, C₁-C₈ thioester, C₂-C₈ ether, C₁-C₈ thioether, amide (amido or carboxamido), substituted amide (especially mono- or di-alkylamide) or alkyleneamide, an optionally substituted carbamate or urethane group, wherein an alkylene group or other carbon group not otherwise specified contains from 1 to 8 carbon units long (alternatively, about 2-6 carbon units long) and the alkyl group on an ester group is from 1 to 8 carbon units long, preferably up to 4 carbon units long, among numerous others. Various optionally substituted moieties may be substituted with 5 or more substituents, preferably no more than 3 substituents and preferably from 1 to 3 substituents.

The term “pharmaceutically acceptable salt” is used throughout the specification to describe a salt form of one or more of the compositions (and in particularly preferred aspects according to the present invention, phosphate salts) herein which are presented to increase the solubility of the compound in saline for parenteral delivery or in the gastric juices of the patient's gastrointestinal tract in order to promote dissolution and the bioavailability of the compounds. Pharmaceutically acceptable salts include those derived from pharmaceutically acceptable inorganic or organic bases and acids. Suitable salts include those derived from alkali metals such as potassium and sodium, alkaline earth metals such as calcium, magnesium and ammonium salts, among numerous other acids well known in the pharmaceutical art. Sodium and potassium salts are particularly preferred as neutralization salts of carboxylic acids and free acid phosphate containing compositions according to the present invention. The term “salt” shall mean any salt consistent with the use of the compounds according to the present invention. In the case where the compounds are used in pharmaceutical indications, including the treatment of neoplasia, including cancer, the term “salt” shall mean a pharmaceutically acceptable salt, solvate or polymorph consistent with the use of the compounds as pharmaceutical agents.

The term “pharmaceutically acceptable derivative” or “derivative” is used throughout the specification to describe any pharmaceutically acceptable prodrug form (such as an ester, ether or other prodrug group such as an amide group) which, upon administration to a patient, provides directly or indirectly the present compound or an active metabolite of the present compound.

The term “cancer” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated. As used herein, the term cancer is used to describe all cancerous disease states and embraces or encompasses the pathological process associated with malignant hematogenous, ascitic and solid tumors. Representative cancers include, for example, stomach, colon, rectal, liver, pancreatic, lung, breast, cervix uteri, corpus uteri, ovary, prostate, testis, bladder, renal, thyroid, brain/CNS, head and neck, throat, Hodgkin's disease, non-Hodgkin's lymphoma, multiple myeloma, leukemia, melanoma, non-melanoma skin cancer, acute lymphocytic leukemia, acute myelogenous leukemia, Ewing's sarcoma, small cell lung cancer, choriocarcinoma, glioblastoma, rhabdomyosarcoma, Wilms' tumor, neuroblastoma, hairy cell leukemia, mouth/pharynx, oesophagus, larynx, kidney cancer and lymphoma, among others, which may be treated by one or more compounds according to the present invention. In certain aspects of the present invention, compounds and methods are particularly effective in treating glioblastoma, melanoma, breast cancer, prostate cancer, ovarian cancer, lung cancer and thyroid cancer by reducing the likelihood of metastatis of the cancer to other areas of the patient's body.

The term “tumor” is used to describe an abnormal growth in tissue which occurs when cellular proliferation is more rapid than normal tissue and continues to grow after the stimuli that initated the new growth cease. The term includes a malignant cancerous growth or tumefacent. Tumors generally exhibit partial or complete lack of structural organization and functional coordination with the normal tissue, and usually form a distinct mass of tissue which may be benign (benign tumor) or malignant (carcinoma). Tumors tend to be highly vascularized. The term “cancer” is used as a general term herein to describe malignant tumors or carcinoma. These malignant tumors may invade surrounding tissues, may metastasize to several sites and are likely to recur after attempted removal and to cause death of the patient unless adequately treated. As used herein, the terms carcinoma and cancer are subsumed under the term tumor. The present compounds may be used to treat tumors, regardless of their malignancy or etiology.

The term “anti-cancer compound” or “anti-cancer agent” is used to describe any compound (including its derivatives) other than the anti-metastatic compounds according to the present invention which may be used to treat cancer. Anti-cancer compounds for use in the present invention may be co-administered with one or more of the compounds of the present invention its derivative compounds have on enhancing the effect of the anti-cancer compound in treating cancer in a patient pursuant to the present invention. In many instances the co-administration of a compound according to the present invention and another anti-cancer compound results in a synergistic anti-cancer effect. Exemplary anti-cancer compounds for use in the present invention for co-administration with compounds according to the present invention include anti-metabolites agents which are broadly characterized as antimetabolites, inhibitors of topoisomerase I and II, alkylating agents and microtubule inhibitors (e.g., taxol), as well as tyrosine kinase inhibitors (e.g., surafenib), EGF kinase inhibitors (e.g., tarceva or erlotinib) and ABL kinase inhibitors (e.g. gleevec or imatinib). Anti-cancer compounds for use in the present invention include, for example, Aldesleukin; Alemtuzumab; alitretinoin; allopurinol; altretamine; amifostine; anastrozole; arsenic trioxide; Asparaginase; BCG Live; bexarotene capsules; bexarotene gel; bleomycin; busulfan intravenous; busulfan oral; calusterone; capecitabine; carboplatin; carmustine; carmustine with Polifeprosan 20 Implant; celecoxib; chlorambucil; cisplatin; cladribine; cyclophosphamide; cytarabine; cytarabine liposomal; dacarbazine; dactinomycin; actinomycin D; Darbepoetin alfa; daunorubicin liposomal; daunorubicin, daunomycin; Denileukin diftitox, dexrazoxane; docetaxel; doxorubicin; doxorubicin liposomal; Dromostanolone propionate; Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemtuzumab ozogamicin; gleevec (imatinib); goserelin acetate; hydroxyurea; Ibritumomab Tiuxetan; idarubicin; ifosfamide; imatinib mesylate; Interferon alfa-2a; Interferon alfa-2b; irinotecan; letrozole; leucovorin; levamisole; lomustine (CCNU); meclorethamine (nitrogen mustard); megestrol acetate; melphalan (L-PAM); mercaptopurine (6-MP); mesna; methotrexate; methoxsalen; mitomycin C; mitotane; mitoxantrone; nandrolone phenpropionate; Nofetumomab; LOddC; Oprelvekin; oxaliplatin; paclitaxel; pamidronate; pegademase; Pegaspargase; Pegfilgrastim; pentostatin; pipobroman; plicamycin; mithramycin; porfimer sodium; procarbazine; quinacrine; Rasburicase; Rituximab; Sargramostim; streptozocin; surafenib; talbuvidine (LDT); talc; tamoxifen; tarceva (erlotinib); temozolomide; teniposide (VM-26); testolactone; thioguanine (6-TG); thiotepa; topotecan; toremifene; Tositumomab; Trastuzumab; tretinoin (ATRA); Uracil Mustard; valrubicin; valtorcitabine (monoval LDC); vinblastine; vinorelbine; zoledronate; and mixtures thereof, among others.

The term “bioactive agent” includes any biologically active agent, including a prodrug form of the active agent, which can be administered in combination with a compound according to the present invention pursuant to the present invention and can include active agents or their derivatives which provide additional biological activity which is shown to be advantageous for a cancer patient. In addition to anticancer agents as otherwise described above, bioactive agents may include a number of antiviral agents including for example, agents which are useful for the treatment of HIV, HBV and other viral infections as well as agents which treat hyperproliferative diseases and chronic inflammatory diseases such as arthritis, including rheumatoid arthritis and osteoarthritis, among numerous others, including analgesic agents, including opioid analgesics, which are helpful in reducing pain in patients with cancer, arthritis and other conditions. In the case of treating cander or angiogenesis related diseases as otherwise disclosed herein, compounds according to the present invention may be combined with an anti-vascular endothelial growth factor agent (anti-VEGF agent), such as a monoclonal antibody such as bevacizumab (Avastin), an antibody derivative such as ranibizumab (Lucentis), or orally-available small molecules that inhibit the tyrosine kinases stimulated by VEGF: sunitinib (Sutent), sorafenib (Nexavar), axitinib and pazopanib agents.

The term “angiogenesis” is used throughout the specification to describe the biological processes which result in the development of blood vessels or increase in the vascularity of tissue in an organism. With respect to the present invention, the term angiogenesis is defined as the process through which tumors or other rapidly proliferating tissue derive a blood supply through the generation of microvessels.

The terms “angiogenic disease”, “angiogenic disorder” and “angiogenic skin disorder” is used throughout the specification to describe a disorder, including a skin disorder or related disorder which occurs as a consequence of or which results in increased vascularization in tissue. Oftentimes, the etiology of the angiogenic disease is unkown. However, whether angiogenesis is an actual cause of a disease state or is simply a condition of the disease state is unimportant, but the inhibition of angiogenesis in treating, ameliorating and/or reversing the disease state or condition is another aspect of the present invention. Examples of angiogenic skin disorders which may be treated utilizing compounds according to the present invention include, for example, macular degeneration, especially including exudative (wet) macular degeneration, diabetic retinopathy, psoriasis, venous ulcers, acne, rosacea, warts, eczema, hemangiomas and lymphangiogenesis, among numerous others, including Sturge-Weber syndrome, neurofibromatosis, tuberous sclerosis, chronic inflammatory disease and arthritis. A benign tumor is also considered an angiogenic disease or condition hereunder. Any skin or other disorder which has as a primary or secondary characterization, increased vascularization, is considered an angiogenic disorder for purposes of the present invention and is amenable to treatment with compounds according to the present invention.

The term “macular degeneration” or “exudative (wet) macular degeneration” is used to describe a medical condition usually of older adults which results in a loss of vision in the center of the visual field (the macula) because of damage to the retina. Macular degeneration occurs in “dry” and “wet” forms. It is a major cause of blindness in the elderly (>50 years). Macular degeneration can make it difficult or impossible to read or recognize faces, although enough peripheral vision remains to allow other activities of daily life.

The inner layer of the eye is the retina, which contains nerves that communicate sight, and behind the retina is the choroid, which contains the blood supply to the retina. In the wet (exudative) form of macular degeneration, which is more severe, blood vessels grow up from the choroid behind the retina, and the retina can also become detached. It can be treated with laser coagulation, and with medication that stops and sometimes reverses the growth of blood vessels. Although some macular dystrophies affecting younger individuals are sometimes referred to as macular degeneration, the term generally refers to age-related macular degeneration (AMD or ARMD). Neovascular or exudative AMD, the “wet” form of advanced AMD, causes vision loss due to abnormal blood vessel growth in the choriocapillaries, through Bruch's membrane, ultimately leading to blood and protein leakage below the macula. Bleeding, leaking, and scarring from these blood vessels eventually cause irreversible damage to the photoreceptors and rapid vision loss if left untreated.

In the present invention, macular degeneration, in particular, exudative (wet) macular degeneration may be treated using one or more compounds according to the present invention alone or in combination with another agent, for example, an anti-vascular endothelial growth factor (anti-VEGF agent), such as a monoclonal antibody such as bevacizumab (Avastin), an antibody derivative such as ranibizumab (Lucentis), or orally-available small molecules that inhibit the tyrosine kinases stimulated by VEGF: sunitinib (Sutent), sorafenib (Nexavar), axitinib and pazopanib.

The term “diabetic retinopathy” is used to describe retinopathy (damage to the retina) caused by complications of diabetes mellitus, which can eventually lead to blindness. It is an ocular manifestation of systemic disease which affects up to 80% of all patients who have had diabetes for 10 years or more.

Diabetic retinopathy often has no early warning signs. Even macular edema, which may cause vision loss more rapidly, may not have any warning signs for some time. In general, however, a person with macular edema is likely to have blurred vision, making it hard to do things like read or drive. In some cases, the vision will get better or worse during the day.

As new blood vessels form at the back of the eye as a part of proliferative diabetic retinopathy (PDR), they can bleed (hemorrhage) and blur vision. The first time this happens, it may not be very severe. In most cases, it will leave just a few specks of blood, or spots, floating in a person's visual field, though the spots often go away after a few hours.

These spots are often followed within a few days or weeks by a much greater leakage of blood, which blurs vision. In extreme cases, a person will only be able to tell light from dark in that eye. It may take the blood anywhere from a few days to months or even years to clear from the inside of the eye, and in some cases the blood will not clear. These types of large hemorrhages tend to happen more than once, often during sleep. In treating diabetic retinopathy, including proliferative retinopathy, compounds according to the present invention may be used alone or in combination with triamcinolone acetonide, preferably administered intravitreally (intravitreal administration). Pharmaceutical compositions based upon a combination of such agents, in combination with a pharmaceutically acceptable carrier, additive or excipient, are also contemplated by the present invention.

The term “rosacea” is used to describe acne rosacea or erythematosa characterized by vascular and follicular dilation involving the nose and continguous portions of the cheeks. Rosacea may vary from very mild but persistent erythema to extensive hyperplasia of the sebaceous glands with deep-seated papules and pustules and accompanied by telangiectasia at the affected erythematous sites. Also called hypertrophic rosacea or rhinophyma, depending upon the severity of the condition.

The term “wart” is used to describe a small, usually hard tumerous growth on the skin. Also known as a verrucas, a wart is a flesh-colored growth of the skin which is characterized by circumscribed hypertrophy of the papillae of the corium, with thickening of the malpighian, granulation and keratin layers of the epidermis. Verucca vulgaris, a subset of warts or verruca, is characterized by infection of the keratinocytes with human papillomavirus.

The term “psoriasis” is used to describe a skin condition which is characterized by the eruption of circumscribed, discrete and confluent, reddish, silvery-scaled maculopapules; the lesions occur preeminently on the elbows, knees, scalp and trunk and microscopically show characteristic parakeratosis and elongation of rete ridges.

The term “acne” is used to describe a condition of the skin characterized by inflammatory follicular, papular and pustular eruptions involving the sebaceous apparatus. Although there are numerous forms of acne, the most common form is known as acne simplex or acne vulgaris which is characeterized by eruptions of the face, upper back and chest and is primarily comprised of comedones, cysts, papules and pustules on an inflammatory base. The condition occurs primarily during puberty and adolesence due to an overactive sebaceous apparatus which is believed to be affected by hormonal activity.

The term “eczema” is a generic term used to describe acute or chronic inflammatory conditions of the skin, typically erythematous, edematous, papular, vesicular, and crusting; followed often by lichenification and scaling and occasionally by duskiness of the erythema and, infrequently, hyperpigmentation. Eczema is often accompanied by the sensation of itching and burning. Eczema vesicles form by intraepidermal spongiosis. Eczema is sometimes referred to colloquially as tetter, dry tetter and scaly tetter. There are numerous subcategories of eczema, all of which are treated by one or more of the compounds according to the present invention.

The term “coadministration” or “combination therapy” is used to describe a therapy in which at least two active compounds in effective amounts are used to treat cancer, inhibit, reduce the likelihood of or prevent growth and metastasis of cancer, inhibition angiogenesis or treat an angiogenic disease state or condition at the same time. Although the term coadministration preferably includes the administration of two active compounds to the patient at the same time, it is not necessary that the compounds be administered to the patient at the same time, although effective amounts of the individual compounds will be present in the patient at the same time. Compounds according to the present invention may be administered with one or more anti-cancer agent as otherwise described herein. Coadministration also embraces the administration of dual analogs (i.e., compounds wherein at least two biologically active compounds are chemically linked via a chemical linker such as, for example, without limitation, phosphate groups or carboxylate groups, among others as otherwise described herein) or other dual antagonists, where at least one of the active compounds of the dual antagonist compound is a compound as otherwise described herein.

In the case of treating tumors and/or cancer or one or more hyperprolifeative diseases as otherwise discussed herein, coadministration may be with one or more anti-cancer agent such antimetabolites, Ara C, etoposide, doxorubicin, taxol, hydroxyurea, vincristine, cytoxan (cyclophosphamide), methotrexate, or mitomycin C, among numerous others, including topoisomerase I and topoisomerase II inhibitors, such as adriamycin, topotecan, campothecin and irinotecan, other agent such as gemcitabine and agents based upon campothecin and cis-platin may be included. These compounds may also be included in pharmaceutical formulations or coadministered with compounds according to the present invention to produce additive or synergistic anti-cancer activity. Exemplary anti-cancer compounds which may be used for co-administrtion with compounds according to the present invention in the treatment of cancer include, for example, Aldesleukin; Alemtuzumab; alitretinoin; allopurinol; altretamine; amifostine; anastrozole; arsenic trioxide; Asparaginase; BCG Live; bexarotene capsules; bexarotene gel; bleomycin; busulfan intravenous; busulfan oral; calusterone; capecitabine; carboplatin; carmustine; carmustine with Polifeprosan 20 Implant; celecoxib; chlorambucil; cisplatin; cladribine; cyclophosphamide; cytarabine; cytarabine liposomal; dacarbazine; dactinomycin; actinomycin D; Darbepoetin alfa; daunorubicin liposomal; daunorubicin, daunomycin; Denileukin diftitox, dexrazoxane; docetaxel; doxorubicin; doxorubicin liposomal; Dromostanolone propionate; Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemtuzumab ozogamicin; goserelin acetate; hydroxyurea; Ibritumomab Tiuxetan; idarubicin; ifosfamide; imatinib mesylate; Interferon alfa-2a; Interferon alfa-2b; irinotecan; letrozole; leucovorin; levamisole; lomustine (CCNU); meclorethamine (nitrogen mustard); megestrol acetate; melphalan (L-PAM); mercaptopurine (6-MP); mesna; methotrexate; methoxsalen; mitomycin C; mitotane; mitoxantrone; nandrolone phenpropionate; Nofetumomab; LOddC; Oprelvekin; oxaliplatin; paclitaxel; pamidronate; pegademase; Pegaspargase; Pegfilgrastim; pentostatin; pipobroman; plicamycin; mithramycin; porfimer sodium; procarbazine; quinacrine; Rasburicase; Rituximab; Sargramostim; streptozocin; talbuvidine (LDT); talc; tamoxifen; temozolomide; teniposide (VM-26); testolactone; thioguanine (6-TG); thiotepa; topotecan; toremifene; Tositumomab; Trastuzumab; tretinoin (ATRA); Uracil Mustard; valrubicin; valtorcitabine (monoval LDC); vinblastine; vinorelbine; zoledronate; and mixtures thereof, among others.

Compounds according to the present invention may also be coadministered with other angiogenesis inhibitors including anti-VEGF compounds or compositions, such as such as a monoclonal antibody such as bevacizumab (Avastin), an antibody derivative such as ranibizumab (Lucentis), or orally-available small molecules that inhibit the tyrosine kinases stimulated by VEGF: sunitinib (Sutent), sorafenib (Nexavar), axitinib and pazopanib.

The present invention includes the compositions comprising the pharmaceutically acceptable salts of compounds of the present invention. The acids which may be used to prepare the pharmaceutically acceptable acid addition salts of the aforementioned compounds useful in this invention are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3 naphthoate)]salts, among numerous others.

The invention also includes compositions comprising base addition salts of the present compounds. The chemical bases that may be used as reagents to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (eg., potassium and sodium) and alkaline earth metal cations (e, calcium and magnesium), ammonium or water-soluble amine addition salts such as N-methylglucamine-(meglumine), and the lower alkanolammonium and other base salts of pharmaceutically acceptable organic amines, among others.

The compositions of the present invention may be formulated in a conventional manner using one or more pharmaceutically acceptable carriers and may also be administered in controlled-release formulations. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Hely or similar alcohol.

The pharmaceutical compositions of this invention may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be administered topically, especially to treat melanoma or other cancers which occur in or on the skin (non-melanoma skin cancer). Suitable topical formulations are readily prepared for each of these areas or organs. Topical application for the lower intestinal tract can be effected in a rectal suppository formulation (see above) or in a suitable enema formulation. Topically-acceptable transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water.

Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with our without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of compound in a pharmaceutical composition of the instant invention that may be combined with the carrier materials to produce a single dosage form will vary depending upon the host and disease treated, the particular mode of administration. Preferably, the compositions should be formulated to contain between about 0.5 milligram to about 750 milligrams, more preferably about 1 milligram to about 600 milligrams, and even more preferably about 10 milligrams to about 500 milligrams of active ingredient. The amount will vary as a function of the agent, its route of administration, the cancer to be treated and the size and age of the patient, among other factors to be considered.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

### Preparation and Administration of the Active Compounds and Compositions

Compounds according to the present invention can be prepared according to typical chemical synthetic methods which are well known in the art by simple analogy with methods which are readily available. One of ordinary skill may readily provide compounds and pharmaceutical compositions as otherwise described herein.

Humans, equines, canines, bovines and other animals, and in particular, mammals, suffering from cancer can be treated by administering to the patient (subject) an effective amount of a compound according to the present invention or its derivative, including a pharmaceutically acceptable salt thereof optionally in a pharmaceutically acceptable carrier or diluent, either alone, or in combination with other known anticancer or pharmaceutical agents. This treatment can also be administered in conjunction with other conventional cancer therapies, such as radiation treatment or surgery.

These compounds can be administered by any appropriate route, for example, orally, parenterally, intravenously, intradermally, subcutaneously, or topically, in liquid, cream, gel, or solid form, or by aerosol form.

The active compound is included in the pharmaceutically acceptable carrier or diluent in an amount sufficient to deliver to a patient a therapeutically effective amount for the desired indication, without causing serious toxic effects in the patient treated. A preferred dose of the active compound for all of the herein-mentioned conditions is in the range from about 10 ng/kg to 300 mg/kg, preferably 0.1 to 100 mg/kg per day, more generally 0.5 to about 25 mg per kilogram body weight of the recipient per day. A typical topical dosage will range from 0.01-3% wt/wt in a suitable carrier.

The compound is conveniently administered in any suitable unit dosage form, including but not limited to one containing 1 to 3000 mg, preferably 5 to 500 mg of active ingredient per unit dosage form. An oral dosage of about 25-250 mg is usually convenient.

The active ingredient is preferably administered to achieve peak plasma concentrations of the active compound of about 0.00001-30 mM, preferably about 0.1-30 μM. This may be achieved, for example, by the intravenous injection of a solution or formulation of the active ingredient, optionally in saline, or an aqueous medium or administered as a bolus of the active ingredient.

The concentration of active compound in the drug composition will depend on absorption, distribution, inactivation, and excretion rates of the drug as well as other factors known to those of skill in the art. It is to be noted that dosage values will also vary with the type and severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

Oral compositions will generally include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or enteric agents.

The active compound or pharmaceutically acceptable salt thereof can be administered as a component of an elixir, suspension, syrup, wafer, chewing gum or the like. A syrup may contain, in addition to the active compounds, sucrose as a sweetening agent and certain preservatives, dyes and colorings and flavors.

The active compound or pharmaceutically acceptable salts thereof can also be mixed with other active materials that do not impair the desired action, or with materials that supplement the desired action, such as other anticancer agents, antibiotics, antifungals, antiinflammatories, antiviral compounds andn analgesic compounds. In preferred aspects of the invention, compounds according to the present invention are coadministered with another anticancer agent, as otherwise described herein.

Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

Liposomal suspensions may also be pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound are then introduced into the container. The container is then swirled by hand to free lipid material from the sides of the container and to disperse lipid aggregates, thereby forming the liposomal suspension.

### Biological Activity

A wide variety of biological assays have been used and are accepted by those skilled in the art to assess anti-cancer, anti-metastatic and anti-angiogenic activity of compounds. A number of these methods as well as the specific methods which are described hereinbelow may be used to evaluate the activity of the compounds disclosed herein.

### EXAMPLES

### Materials and Methods

Reagents: All chemicals and reagents were the highest purity commercially available. p-Nitrophenyl 5′-thymidine monophosphate (pNP-TMP) was purchased as a dry powder from Sigma (St. Louis, Mo.). Fluorescent Substrate-3 (FS-3) came from Echelon (Salt Lake City, Utah). Both substrates were freshly dissolved in assay buffer (50 mM Tris, 5 mM KCl, 140 mM NaCl, 1 mM MgCl₂, and 1 mM CaCl₂, pH 8.0) immediately before use. RJC 03297 was purchased from Maybridge, and NSC 48300 was obtained from the NCI Developmental Therapeutics Program (DTP) Open Chemical Repository (see, http://dtp.nci.nih.gov/). The structure and purity (95%) of NSC 48300 was confirmed by ¹H NMR and mass spectroscopy. Analogues of NSC 48300 were identified by performing a substructure search against the NCI Open Chemical Repository collection. Analogues deemed useful for establishing structure-activity relationships were identified and individually requested from the DTP. Hexachlorophene, merbromin, bithionol, 2′2′-Methylenebis(4-chlorophenol), and Eosin Y were purchased from Sigma and all inhibitors were solubilized at 10-100 mM in DMSO for steady-state kinetics and DMSO (hexachlorophene, bithionol, 2′2′-Methylenebis(4-chlorophenol), and NSC 48300) or water (merbromin) for cell motility assays.

Cell culture: Human melanoma cells, A2058 (ATCC), were grown in Dulbecco's Modified Eagle's Medium (DMEM) (Gibco), MeI 28 (ATCC), YUSAC2 (Yale University) and MeI-888 (Yale University) were grown in OptiMEM media (Gibco), and HTB 63 (ATCC) were grown in McKoy's 5a medium (ATCC). Human breast adenocarcinoma cells, MDA-MB-231(ATCC) and ovarian carcinoma cells R182 (Gil Mor, Yale University) were grown in RPMI-1640 (Gibco). All cell media was supplemented with 10% (v/v) fetal calf serum (FCS) (Gibco) and 1% (v/v) L-glutamine (Gibco) and grown at 37° C. in a humidified atmosphere containing 5% CO₂.

Protein expression and purification: The full length human NPP2 gene (NCBI accession #BC034961) followed by a C-terminal TEV cleavage site and a 9-His and 6-His purification tag was cloned and transferred into a p-DEST-8 baculovirus shuttle vector (Invitrogen), and bacmid DNA was generated by standard methods. The DNA sequence and correct transposition of the genes was verified by PCR, and recombinant full-length human ATX was expressed in High Five insect cells using standard methods [34]. Three days post infection, the supernatant was adjusted to a final concentration of 50 mM Tris pH 8.0, 5 mM CaCl₂, and 1 mM NiSO₄ and stirred for 10 min. at room temperature. The resulting precipitant was removed by centrifugation (4,420×g for 30 minutes) and filtration (0.45 μM cutoff), and the supernatant was concentrated using a Pall concentration system to approximately 250 mL. The cell media was passed over a nickel affinity column (5 mL, Fastflow resin), equilibrated in binding buffer (20 mM Tris pH 8.0, 300 mM NaCl, 20 mM imidazole, and 20% ethylene glycol) at 4° C., washed with 10 column volumes of binding buffer, and then eluted with binding buffer supplemented with 300 mM imidazole. Purified ATX was concentrated to approximately 10 mg mL⁻¹, and dialyzed into the appropriate assay buffer.

Polyclonal antibody production and staining: Polyclonal antibodies to recombinant human ATX were generated in chickens by ProSci incorporated (Poway, Calif.). Antibodies were purified with eggcellent chicken IgY purification kit (PIERCE). Tissue microarrays containing 10 examples each of normal skin, nevi, primary melanoma, and metastatic melanoma were obtained from the Yale Tissue bank, and the slides were stained using a 1:1000 dilution of purified antibody.

Inhibitor screen: High throughput screens were performed against two libraries: the GenPlus library of 960 compounds (NINDS Custom Collection, MicroSource Discovery systems) and the NCI Diversity Set (1990 compounds, see http://dtp.nci.nih.gov/branches/dscb/repo_open.html). The screens were performed at the Yale Center for Chemical Genomics utilizing a Tecan Aquarius robot in combination with an Aquarius liquid handler and a Freedom EVO Workstation. A V&P Scientific 384 pin tool transferred small molecules (10 mM stock) from library plates into the optically clear, 384 well assay plates, and an Aquarius liquid handling robot transferred protein into the individual wells. The assays were performed in a total volume of 50 μL in assay buffer (50 mM Tris, 140 mM NaCl, 1 mM MgCl₂, and 5 mM CaCl₂, pH 8.0). Absorbance of p-nitrophenylate, the product of ATX's PDE activity with pNP-TMP (discussed below) was measured at 405 nm over a period of 12 hours. To monitor lysophospholipase-D activity, the fluorescence of FS-3 (625 nM or 4 μM) was monitored (λₑₓ=480 nm, λₑₘ=535 nm) in assay buffer over a period of 30 minutes in the presence of 300 nM ATX and 10 μM compound. The assay was evaluated using a statistical Z′ analysis [35], a quantitative measure to determine assay robustness for single point analysis on a high throughput screening platform. The Z′ analysis was performed from 32 maximum control values (c+) and 32 minimum controls (c−) on each plate. Assays consistently performed with Z′ factors of 0.75-0.85, higher than the minimum 0.5 considered robust.

Steady-state enzyme assays: Phosphodiesterase activity of ATX (75-100 nM) was measured by absorbance (λ=405 nm) on a SpectraMax 250 plate reader at 25° C. with the substrate pNP-TMP [36]. Reactions were run in assay buffer containing 10% DMSO (v/v) and 1 mg mL⁻¹ BSA (hexachlorophene, merbromin, and bithionol) or without BSA (NSC 48300, 2,2′-Methylenebis(4-chlorophenol), eosin Y, and RJC 03297) in a total volume of 150 pt. The uncatalyzed reaction rate was ≦10⁻⁷ s⁻¹ (data not shown).

LysoPLD activity of ATX (75-225 nM) was measured using the fluorescent LPC analogue, FS-3 [36]. Fluorescence was measured (λₑₓ=485 nm, λₑₘ=520 nm) on a SpectraMax Gemini XPS at 37° C. under buffer conditions similar to the PDE assays but lacking BSA. Fluorescence intensity was corrected for inner filter effects [37] using fluorescein. The spontaneous reaction rates were subtracted from the enzyme catalyzed rates.

The steady-state rates (v) of product formation were obtained by fitting the time courses of absorbance or fluorescence change to linear functions. Absorbance units were converted to p-nitrophenylate concentration using ε₄₀₅ of 18.5 mM⁻¹ cm⁻¹ [39] and a measured path length of 0.494 cm. FS-3 hydrolysis product formation was quantitated in arbitrary fluorescence units.

The Michaelis constants (K_{M}) and the catalytic turnover rate constants (k_{cat}) were obtained by fitting the [substrate]-dependence of v to a rectangular hyperbola in the form of the Briggs-Haldane equation:

$$\frac{v}{{[ E ]}_{tot}} = \frac{{k}_{cat} [ S ]}{{K}_{M} + [ S ]} \qquad ( Eq . 1 )$$

in which [S] is [pNP-TMP] or [FS-3]. Inhibitors were treated as reversible inhibitors and analyzed according to the general steady-state linear enzyme inhibitor scheme:

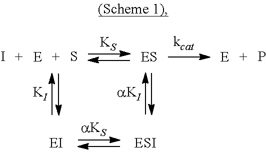

in which E is ATX, S is pNP-TMP or FS-3 substrate, P are the hydrolysis products, and K₁ is the dissociation equilibrium constant for inhibitor binding. The values of α and K₁ were obtained by fitting the steady-state rates of product formation (v) to the general inhibition equation:

$$\frac{v}{{[ E ]}_{tot}} = \frac{{k}_{cat} [ S ]}{{K}_{S} ( 1 + \frac{[ I ]}{{K}_{I}} ) + [ S ] ( 1 + \frac{[ I ]}{α {K}_{I}} )} \qquad ( Eq . 2 )$$

in which [I] is the inhibitor concentration, k_{cat} is the maximum catalytic turnover rate, and K_{S} is the Michaelis constant of the substrate obtained in the absence of inhibitor.

The type of inhibition (competitive, noncompetitive, or uncompetitive) depends on the value of α. Competitive inhibitors have α>>1 and thus fit the equation:

$$\frac{v}{{[ E ]}_{tot}} = \frac{{k}_{cat} [ pNP - T M P ]}{{K}_{S} ( 1 + \frac{[ Hexachlorophene ]}{{K}_{I}} ) + [ pNP - T M P ]} \qquad ( Eq . 3 )$$

Noncompetitive inhibition is the special case in which α=1, which fits the equation:

$$\frac{v}{{[ E ]}_{tot}} = \frac{{k}_{cat} [ pNP - T M P ]}{( {K}_{S} + [ pNP - T M P ] ) ( 1 + \frac{[ Merbromin ]}{{K}_{I}} )} \qquad ( Eq . 4 )$$

Mixed inhibition occurs when alpha differs slightly (<10-fold) from unity so that a single inhibition type does not dominate the effect and the general inhibition equation is used. Uncompetitive inhibition was not observed in this study. The type of inhibition was assigned if the difference between the r² values of the fits to the general inhibition equation (Eq 2) and the specific inhibition (i.e. competitive, noncompetitive or uncompetitive) equations was less than 0.01.

Cell motility assays: Microchemotaxis chambers were employed in Boyden Chamber cell migration assays on a human melanoma cell line (A2058) known to display LPA-dependent chemotaxis [38]. The assay chambers are divided by a gelatin-coated membrane with 8 μM pores (NeuroProbe). Chemo-attractant (ATX ±inhibitor) and/or controls were placed in the lower half of the chamber, and the cells in the upper chamber. The chambers were then incubated at 37° C. for 4 hours, after which the filters were removed, fixed in 100% methanol, stained with a DiffQuick solution, and mounted onto glass slides. Non-migrating cells were removed from the membrane by scraping the top surface. The cells were quantitated by counting the cell number per high powered field in the various wells. Treatments were compared using a chi squared test.

Cell invasion assays: Matrigel matrix (1 mg/ml, Becton Dickenson) was added to 8 μM pore gelatin-coated membranes placed in cell culture inserts (BD Falcon) and allowed to set for 24 hours at room temperature. The inserts were placed into companion 24 well plates (BD Falcon), and chemo-attractants (ATX ±inhibitors) and/or controls were added to the bottom of the wells. Cells (1×10⁶) were added to the inserts and incubated for 3 hours at 37° C. Following incubation, the inserts were removed, fixed in 100% methanol, stained with DiffQuick solution, and mounted on glass slides. Non-invading cells were removed by scraping away the matrigel, and migrating cells were quantitated by counting cell number per high-powered field.

### RESULTS

Purification of ATX. Purified ATX migrates as a single band of ˜100 kDa by SDS-PAGE. The ATX amino acid sequence predicts a mass of 99 kDa after cleavage of the signal peptide and protease cleavage. The mass of purified ATX assayed by Maldi-TOF mass spectrometry is 104,133 Da, consistent with a mature, glycosylated form of the enzyme [41].

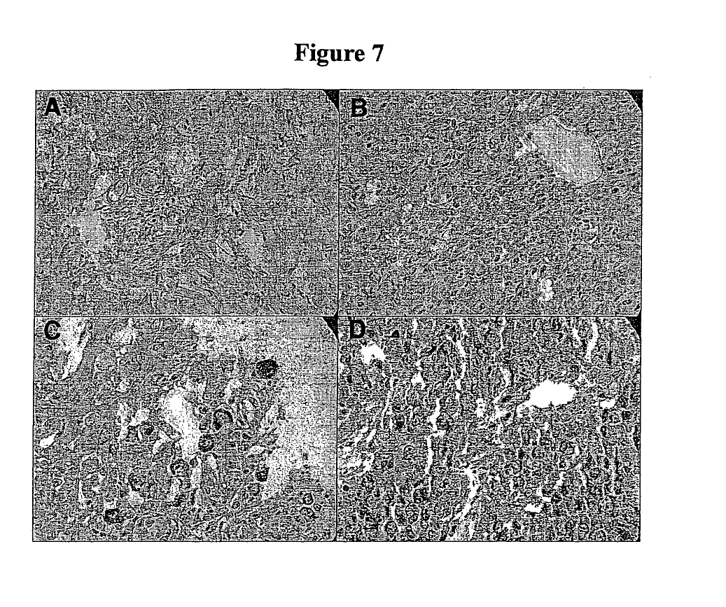

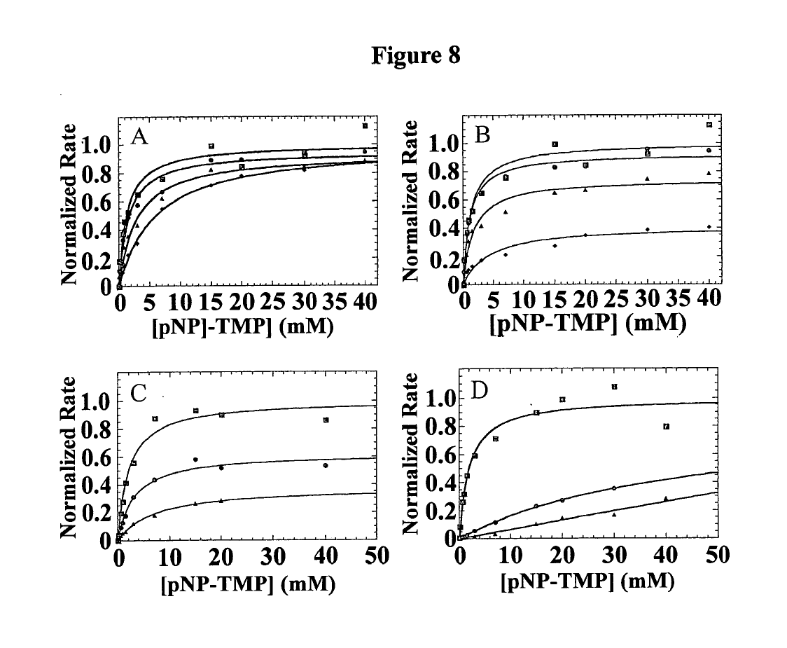

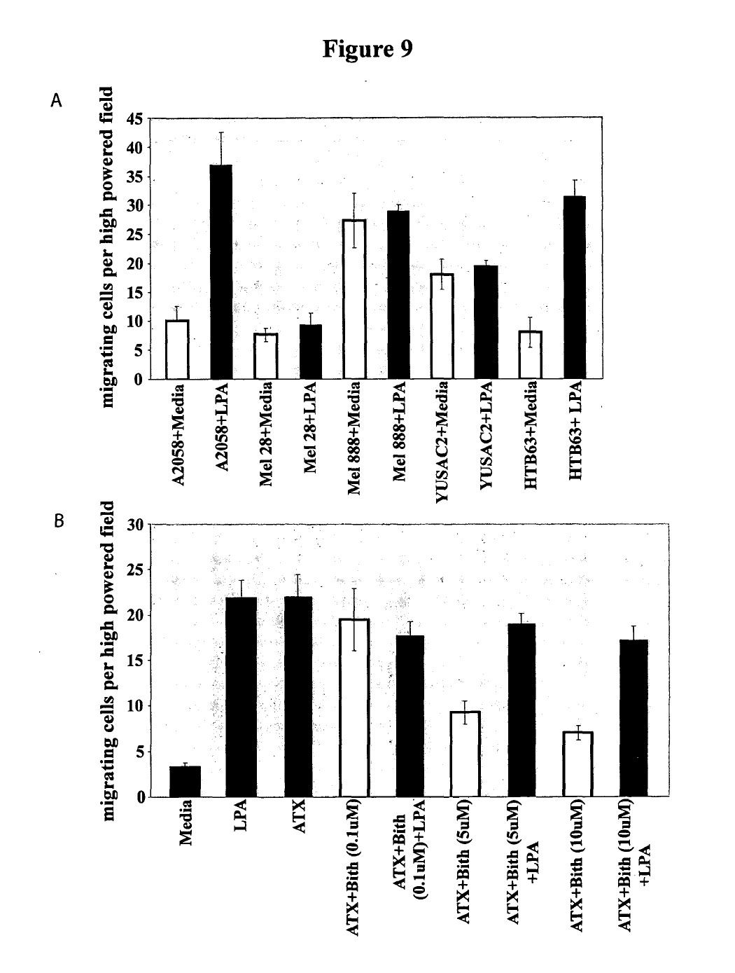

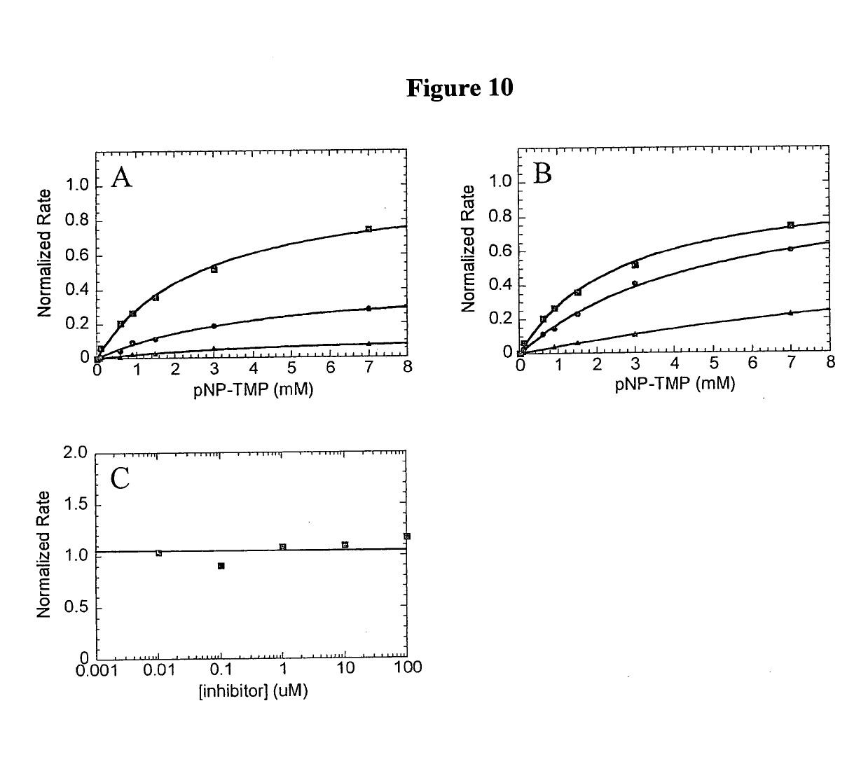

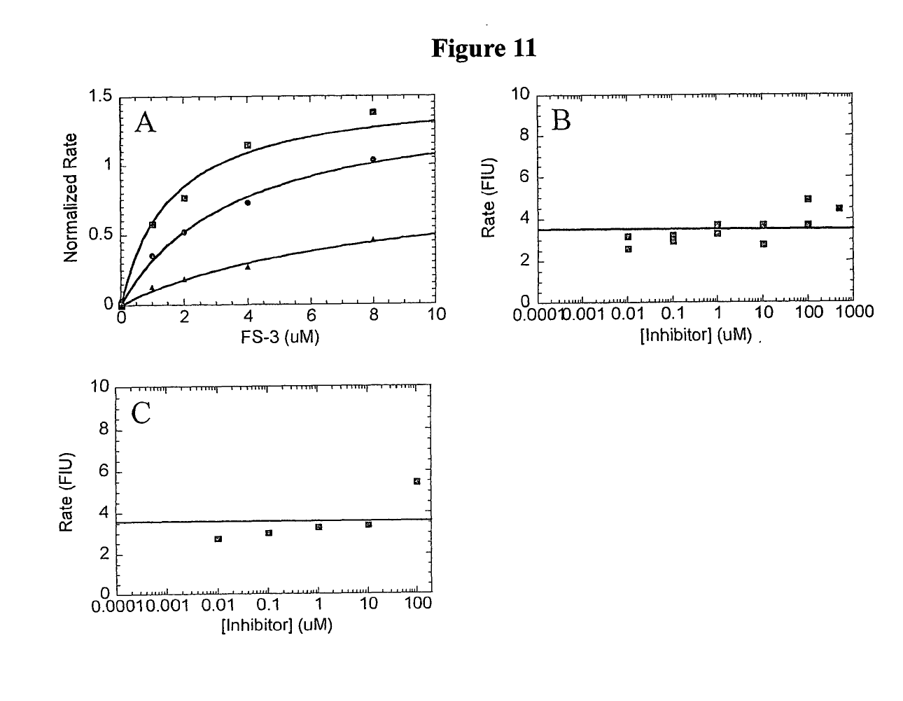

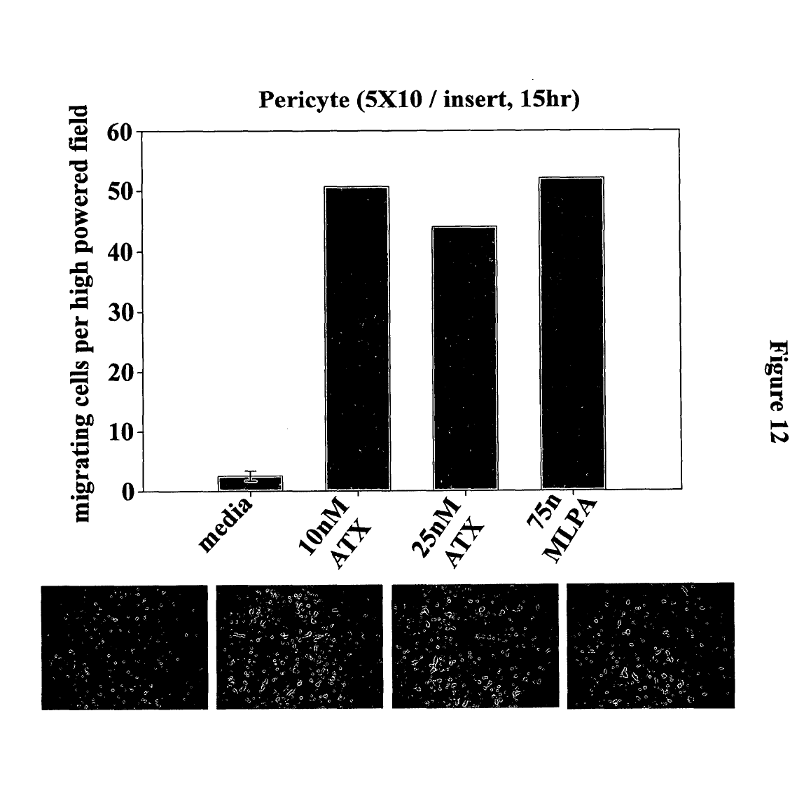

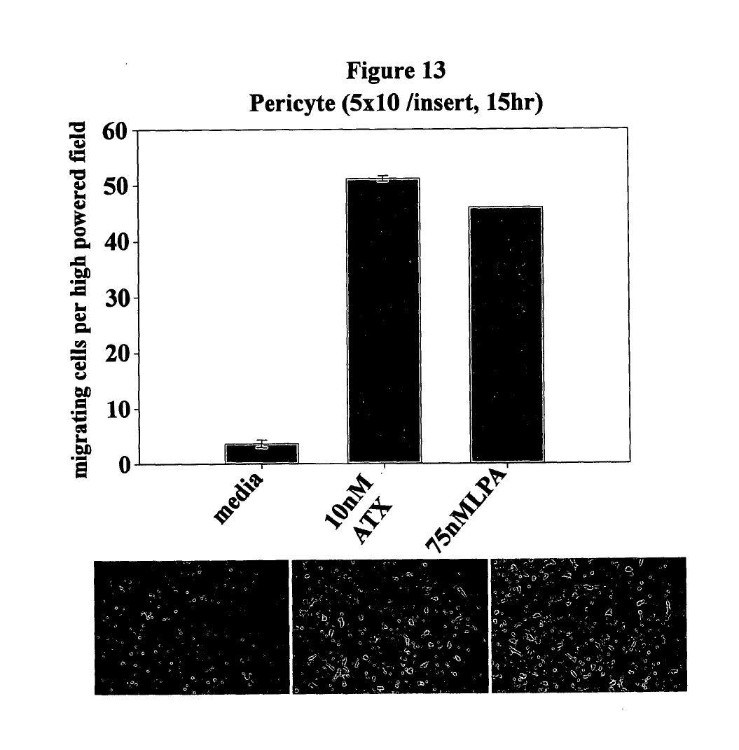

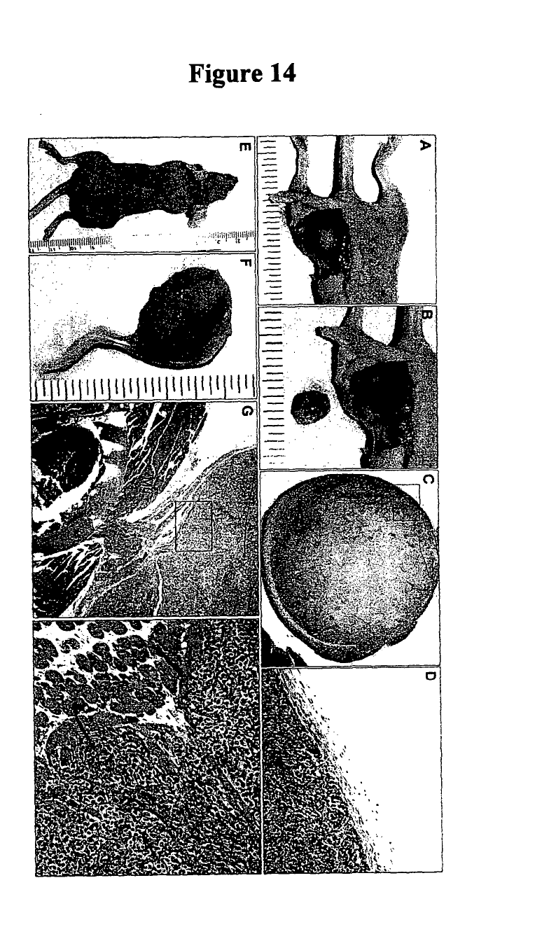

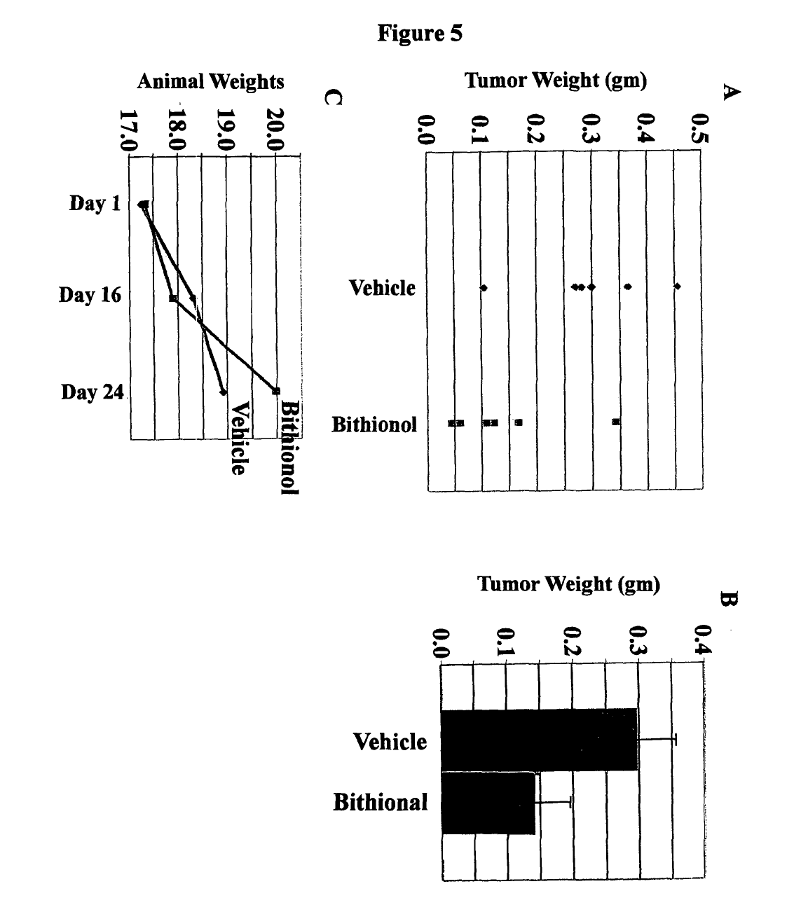

Autotaxin expression in normal skin, benign nevi, and malignant melanoma. The overexpression of ATX in a variety of human malignancies is well established by multiple complementary techniques (see introduction). We sought immunohistochemical confirmation of ATX overexpression in human melanoma using polyclonal antibodies raised against recombinant ATX protein. Human tissue microarrays containing 10 examples of normal skin, benign nevi, primary melanoma, or metastatic melanoma were stained with polyclonal ATX antibodies. Normal skin and benign nevi exhibit no increased expression of ATX, whereas 20% (2 in 10) of the primary and metastatic melanoma sections display strong cytoplasmic reactivity for the protein (FIG. 7).

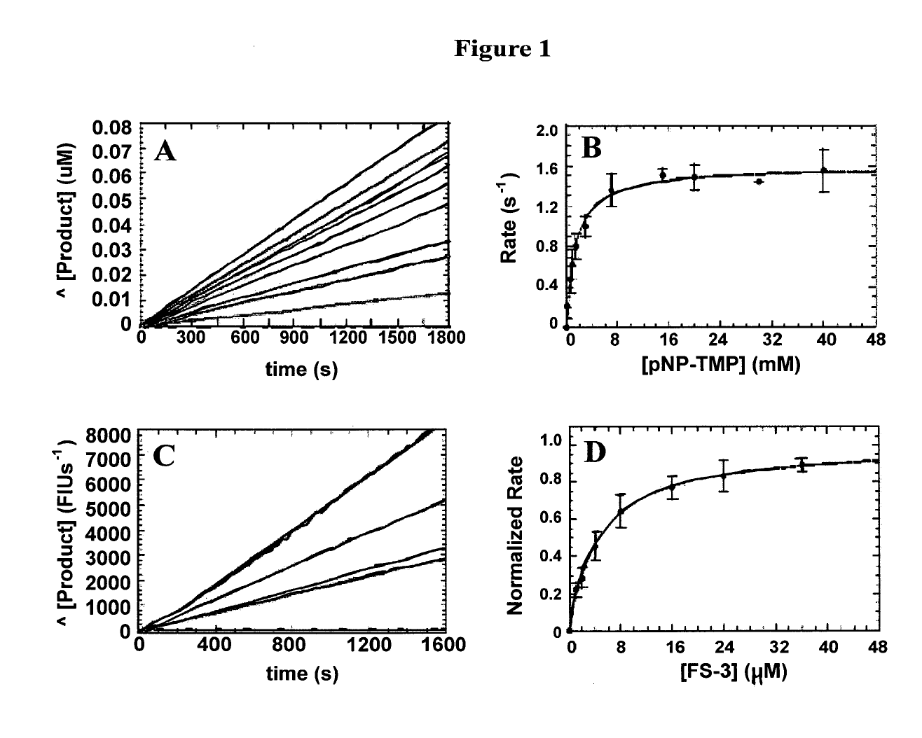

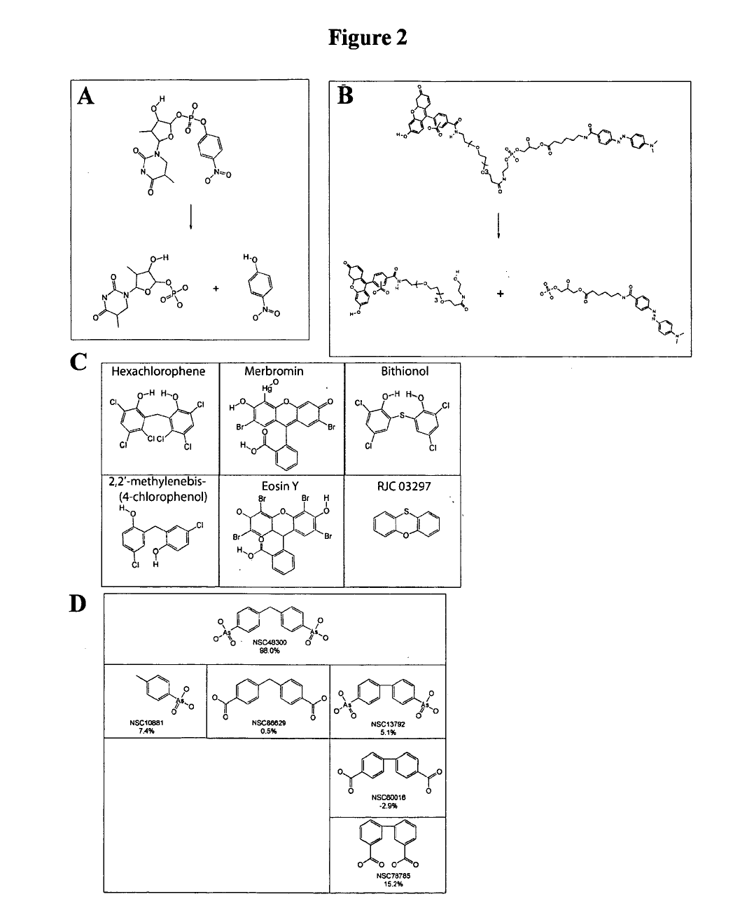

Steady-state enzymatic activity of ATX. The nucleotide PDE activity of ATX was measured using pNP-TMP [36], a modified nucleotide with a phosphodiester bond that is hydrolyzed by ATX to yield p-nitrophenylate (FIG. 2A). Time courses of ATX-dependent pNP-TMP hydrolysis are linear over the timescale measured (30 min; FIG. 1A). The hydrolysis rate (v) depends hyperbolically on the pNP-TMP concentration (FIG. 1B). The best fit of the data to Eq. 1 yields a K_{M} of 1.4 (±0.1) mM and a k_{cat} of 1.6 (±0.1) s⁻¹.

ATX's lysoPLD activity was measured using the fluorescent LPC analog FS-3 as the substrate (FIG. 2B, [36, 37]). Time courses of FS-3 hydrolysis are linear over the timescale examined (15 minutes; FIG. 1C). The hydrolysis rates follow Michaelis-Menton kinetics and depend hyperbolically on the FS-3 concentration (FIG. 1D). The best fit of the data to Eq. 1 yields a K_{M} of 4.5 (±0.6) μM.

Assay conditions were examined to insure that inhibitory affects were not due to loss of ATX's required metal ions. Changes in ATX activity as a result of both Ca²⁺ and Mg²⁺ were measured with pNP-TMP and FS-3 substrates. The apparent metal binding affinities with both substrates are <5 nM (data not shown). Based on our binding affinities for Mg²⁺ and Ca²⁺ as well as published metal affinities for hexachlorophene and bithionol, inhibitors that chelate metal ions (discussed below, [39]), enough metal ions remained in the assay buffer for full ATX activity. Thus, metal chelation is not the cause of the observed inhibition.

Inhibitor screen. We hypothesized that the enzyme would be amenable to small molecule inhibition given the nature of the products and reactants of ATX, and therefore small molecule libraries were screened in a high throughput manner using robotics available at the Yale Chemical Genomics Facility. Initial screens used either pNP-TMP or FS-3 (monitoring the PDE and lyso-PLD activity of the enzyme, respectively) as the substrate as a single active site is reportedly responsible for both activities [2]. The GenPlus library high throughput screen yielded the three hits: hexachlorophene, bithionol, and merbromin (FIG. 2). The NCI Structural Diversity set yielded 10 hits (FIG. 2). We confirmed inhibition by the three GenPlus hits and NSC 48300 from the NCI collection with purified ATX and evaluated effects on cells.

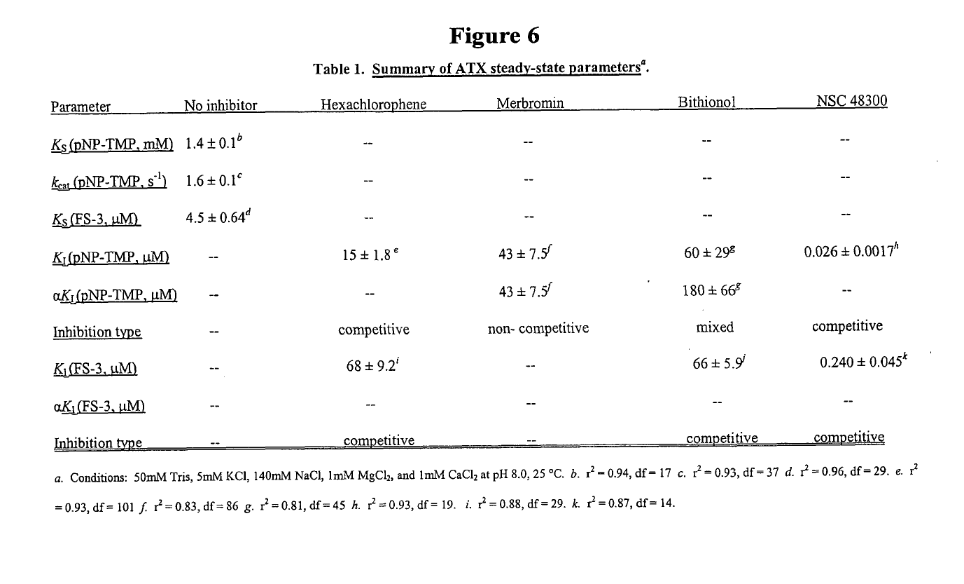

Inhibitory effects on PDE activity of ATX Hexachlorophene (FIG. 2C) slows autotoxin activity in a concentration-dependent manner (Supplemental FIG. 2A). The apparent pNP-TMP K_{M} value increases with hexachlorophene concentration while the k_{cat} value is not significantly affected (Supplemental FIG. 2A). The best fit of the data to the general steady-state equation (Eq. 2) yields α>45, consistent with competitive inhibition of pNP-TMP binding. The hexachlorophene affinity (K_{I}) obtained from the best fit of the data to the competitive inhibition equation (Eq. 3) is 15 (±2) μM (Table 1, FIG. 6).

Merbromin (FIG. 2C) also inhibits autotaxin activity in a concentration-dependent manner. The apparent k_{cat} decreases with increasing merbromin while the apparent K_{S} is unaffected, indicative of noncompetitive inhibition (Supplemental FIG. 2B). The best fit of the data to the noncompetitive inhibition equation (Eq. 4) yields a merbromin K₁ value of 43 (±8) μM (Table 1).

Bithionol (FIG. 2C) inhibits pNP-TMP hydrolysis in a [bithionol]-dependent manner. The apparent pNP-TMP K_{M} value increases with bithionol concentration and the apparent k_{cat} decreases with bithionol, consistent with a mixed, noncompetitive inhibition mechanism (Supplemental FIG. 2C). The K₁ for bithionol binding to ATX calculated from the best fit of the data to the general steady-state inhibition equation (Eq. 2) is 60 (±29) μM and the α value is 3.0 (±2.3) (Table 1).

NSC 48300 (FIG. 2D) inhibits autotaxin activity in a concentration-dependent manner. The apparent K_{M} increases in a [NSC 48300]-dependent manner, while the apparent k_{cat} is unaffected, indicative of competitive inhibition (Supplemental FIG. 2D). The K₁ for NSC 48300 binding ATX calculated from the best fit of the data to the competitive inhibition equation (Eq. 3) is 47.5 nM (Table 1).

Analogs of hexachlorophene, merbromin, and bithionol were identified based on structural motifs (FIG. 2F-H). The hexachlorophene analog, 2,2′-Methylenebis(4-chlorophenol) (FIG. 2C), inhibits ATX noncompetitively with a K₁ of 104 (±15) μM. EosinY (FIG. 2C), the merbromin analog, inhibits ATX competitively with a K₁ of 9.6 (±1.8) μM. RJC 03297 (FIG. 2C), the bithionol analog, does not inhibit ATX at concentrations up to 100 μM.

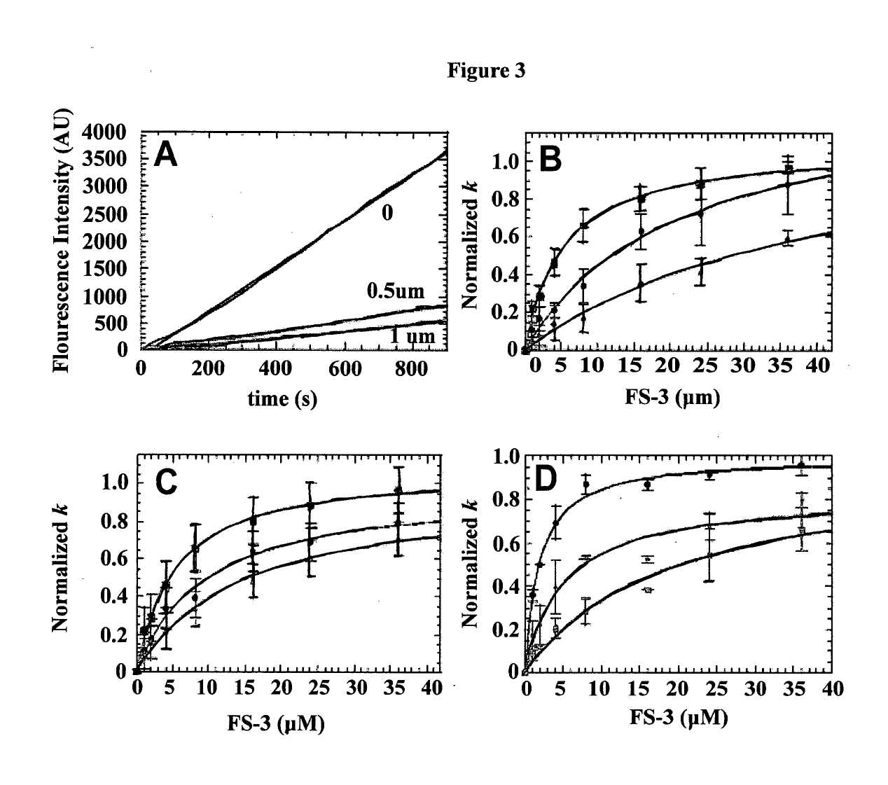

Inhibition of ATX lysoPLD activity. The ability of these compounds to inhibit the lysoPLD activity of ATX was evaluated using FS-3. Hexachlorophene is a competitive inhibitor of ATX lysoPLD activity (FIG. 3B) with a K_{I} value of 68 (±9) μM (Table 1). Bithionol competitively inhibits ATX lysoPLD activity (FIG. 3C), with a K₁ of 66 (±6) μM (Table 1). The similarity between the K₁ values with the pNP-TMP and FS-3 substrates (60 and 66 μM, respectively) is consistent with traditional competitive inhibition since the K₁ reflects binding to free ATX and should be independent of the substrate. NSC 48300 (FIG. 2D) competitively inhibits ATX lysoPLD activity (FIGS. 3A and 3D) with a K_{I} value of 240 (±45) nM (Table 1). Among the GenPlus screen inhibitor analogs (Eosin Y, 2,2′-Methylenebis(4-chlorophenol), RJC 03297), only Eosin Y (an analogue of merbromin) inhibits ATX lysoPLD activity. Inhibition is noncompetitive, with a K₁ of 116 (±37) μM. Merbromin interferes with the assay's fluorescent signal (data not shown), so its effect on lysoPLD activity could not be determined.

Inhibition of ATX lyso-PLD activity by several NSC48300 analogs was qualitatively compared in a time course assay by measuring the production of fluorescent product in the presence or absence of 10 □M inhibitor over a 30 minute interval. The parent compound was found to have nearly complete inhibition with 98.0% less product formation when compared to the enzyme alone. Deletion of an arylarsonic acid and the connected benzene ring weaken inhibition (NSC 10881, FIG. 2D). Substituting the arylarsonic acids with carbonic acids (NSC 86629, FIG. 2D) or removal of the linker, so the benzene rings are directly bound (NSC 13792, FIG. 2D) eliminates inhibition. In the absence of the linker, replacing the arylarsonic acids with carbonic acid again eliminates inhibition, but inhibition can be partially recovered by moving the carbonic acid groups to the meta position.

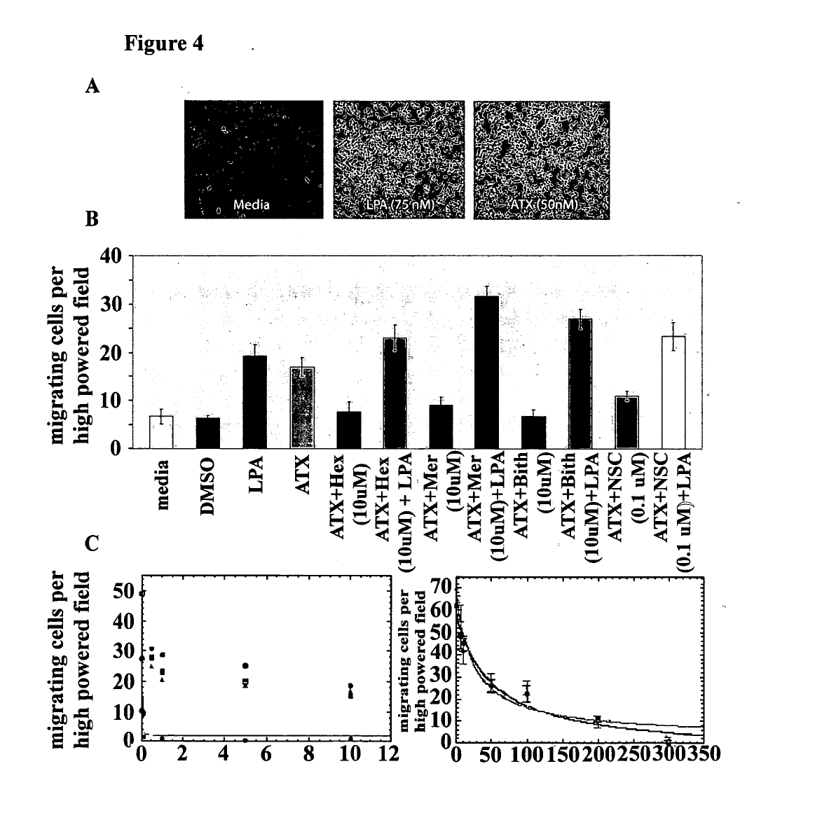

ATX-induced melanoma cell motility and invasion and its inhibition. To verify that the ATX inhibitors were capable of impacting cell motility and invasion, we performed Boyden chamber cell migration assays and matrigel cell invasion assays with melanoma, breast, and ovarian cancer cells. ATX (50 nM) or LPA (75 nM) greatly stimulates A2058 melanoma cell migration in Boyden chamber assays and matrigel invasion assays (FIGS. 4 & 5). The number of migrating cells per high powered field (hpf) increases >20-fold over media alone, and the number of invading cells per hpf increases by 3.5 fold. DMSO (final concentration of 0.3%) does not affect melanoma cell motility or invasion (FIGS. 4A and 4B).

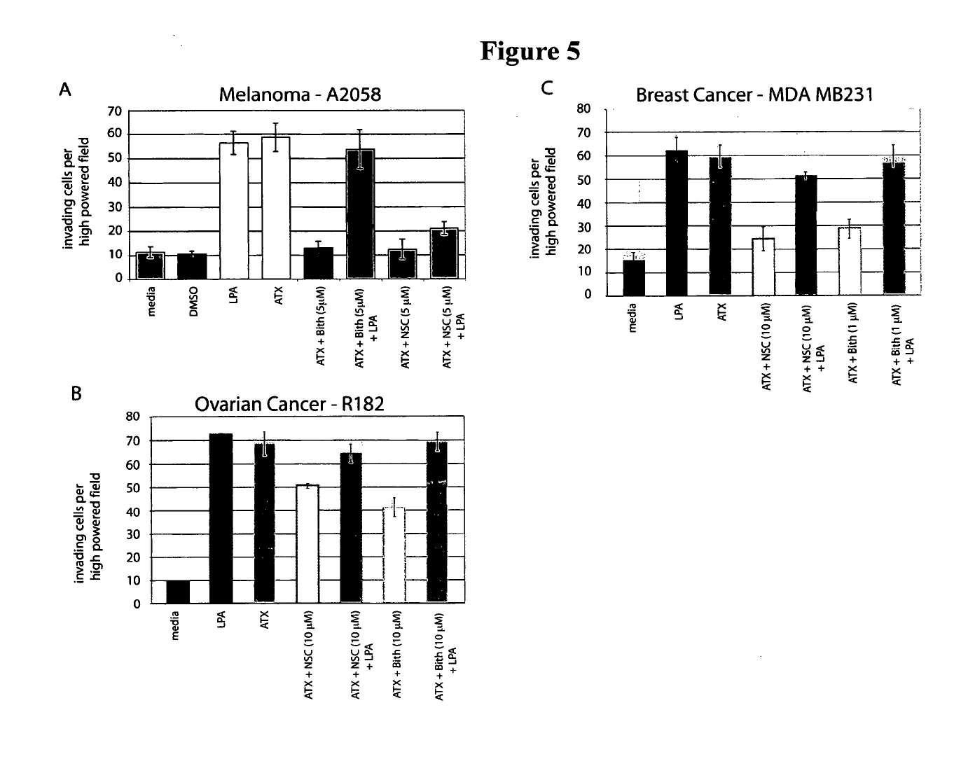

The ATX-induced stimulation of motility and invasion is inhibited in a dose-dependent manner by the ATX inhibitors hexachlorophene, bithionol, merbromin, and NSC 48300. Normal levels of motility and invasion in the presence of inhibitors are regained with the addition of 75 nM LPA (FIG. 4B and FIG. 5) verifying that the cells are still motile, but that their reduced motility is secondary to an absence of LPA. The reduction in motility and invasion by some of the inhibitors could not be fit to a single binding event, as expected from the solution kinetics. This observation is explored in more detail in the discussion below.

Blebbistatin inhibition of ATX-induced cell motility could be experimentally modeled to a single high affinity site resulting in an EC₅₀ of 54 consistent with EC₅₀ values of ˜50 reported for blebbistatin inhibition of adenocarcinoma cell motility [40] (FIG. 4D). The inhibitory effects of blebbistatin could not be recovered by the addition of LPA (EC₅₀=27 μM, FIG. 4D), suggesting that blebbistatin inhibits cell motility downstream of LPA.

To determine if the stimulation of melanoma migration was specific to a single melanoma cell line, we performed cell migration assays on a panel of melanoma cell lines in the presence and absence of the enzymatic product of ATX-LPA. Two of the five cell lines were responsive to LPA stimulation (supplemental FIG. 3A)—A2058 and HTB63. ATX also stimulates HTB63 cell migration, which can be inhibited by bithionol and rescued by excess LPA (supplemental FIG. 3B), identical to the response of the A2058 cells.

To extend these findings to other tumor types and to exclude a cell line dependant artifact, breast cancer cell lines and primary ovarian cancer cells were also tested for ATX stimulation of invasion and an ATX inhibitor effect (FIG. 5). ATX markedly stimulated breast, melanoma, and ovarian cancer cell invasion, an effect which could be reduced with the ATX inhibitors bithionol and NSC48300. The ATX-enhanced invasiveness of the cancer cells in the presence of the inhibitors could be rescued with the addition of LPA, the enzymatic product of ATX, supporting the notion that the inhibitory phenotype was linked to the ATX/LPA axis.

In further experiments, assays were conducted and showed that ATX stimulates pericyte migration, which is a key process in the formation of mature and stable angiogenesis. The cell migration assays were assays in which pericytes are plated on one side of a membrane and either ATX or LPA is added to the other side, and the migration of the cells across a membrane with 8 uM pores is quantified. The results of the assays, which is set forth in FIGS. 10-11, attached, evidences that ATX enhances angiogenesis.

### DISCUSSION

The morbidity and mortality associated with melanoma is linked to its predisposition to metastasize. The prometastatic enzyme autotaxin was initially identified in melanoma cell culture and is associated with tumor aggression, invasion, and growth and metastasis. ATX produces extracellular LPA which binds to G protein coupled receptors at the cell membrane and stimulates cell motility through the phosphoinositide 3-kinase pathway [41], a major signaling cascade deregulated in melanoma. Because of its ubiquity and extracellular location, it is an attractive molecular target in the prevention of metastatic melanoma. To determine whether the enzyme would be a tractable molecular target in the suppression of melanoma invasion and growth and metastasis, we expressed and purified recombinant human protein and used this reagent to develop polyclonal antibodies and to identify small molecule inhibitors. Our results indicate that expression of autotaxin is increased in primary and metastatic melanoma relative to benign skin and nevi, and that small molecule ATX inhibitors suppress melanoma migration and invasion.

Tissue microarrays of normal skin, nevi, and primary and metastatic melanoma stained with chicken polyclonal ATX antibodies demonstrate strong tissue overexpression in a portion (20%) of the primary and metastatic melanoma sections as compared to benign nevi and normal skin (FIG. 1). While clinical data correlating autotaxin expression with melanoma progression is not yet available, ATX expression has been linked to aggressive breast cancer [20] and future immunohistochemical studies with melanoma tissue linked to clinical data may shed light on the correlation between ATX and metastatic melanoma.

Two limited high throughput screens identified viable small molecule leads with affinities from low μM to low nM (FIG. 2, Table 1); analogs of three of these molecules were also evaluated. These seven small molecule inhibitors modulate the PDE enzymatic activity of ATX by competing with substrate binding to free ATX (i.e. they are competitive inhibitors). Six of these inhibitors, with the exception of 2,2′-Methylenebis(4-chlorophenol), also display this behavior with the lysoPLD activity. Bithionol, merbromin, and 2,2′-Methylenebis(4-chlorophenol) additionally display inhibitory activity by binding the ATX-pNP-TMP complex (i.e. they display uncompetitive, noncompetitive, and/or mixed inhibition). Some inhibitors (bithionol, eosin Y, 2,2′-Methylenebis(4-chlorophenol)) bind either the ATX-FS-3 complex or the ATX-pNP-TMP complex, but not both. Hexachlorophene and NSC 48300 competitively inhibit PDE and lysoPLD activities with different K₁ values, unexpected in true competitive inhibitors that bind free enzyme, suggesting formation of a temporary ATX-substrate-inhibitor complex. Evaluation of multiple NSC 48300 analogs suggest that large, electronegative groups below the plane of the greasy center of the molecule are essential for inhibition.

All four inhibitors reduce melanoma cell migration in a dose dependent fashion when assayed using the in vitro cell migration and invasion systems. Base levels of melanoma motility are recovered by the addition of LPA, supporting the notion that the inhibitors decrease tumor motility and invasion by decreasing ATX's lyso-PLD activity and not through non-specific toxic effects on the cells or secondary effects on proteins outside the ATX/LPA pathway. Furthermore, the demonstration that the ATX inhibitors reduce cell invasion in breast and ovarian cancer cells suggests a role for ATX in other malignancies where ATX expression is reported to be increased, and demonstrates that these effects are not an artifact of a particular cell line or tumor type.

Inhibition of cell motility and invasion is biphasic (FIG. 4C and FIG. 5), with some (˜40%) reduction occurring at inhibitor concentrations lower than those needed to modulate ATX activity in solution (FIGS. 3A-D and FIG. 4); complete inhibition occurs over a range comparable to that needed to inhibit ATX (i.e. with apparent K₁ in μM range). We interpret this behavior to reflect the differential partitioning of ATX among the lipid and aqueous phases, which yield deviations from solution steady-state behavior [42]. We can eliminate the possibility that this behavior is non-specific and arises from an LPA/ATX-independent pathway because the inhibitors do not affect the cell motility of tumor cells unresponsive to ATX and LPA stimulation (e.g. mouse melanoma cell line B16, Ouellette and Braddock, unpublished observation). The inhibition by blebbistatin displays the expected kinetics for a soluble enzyme/substrate system, proving that the kinetic behavior discussed above is not an artifact of the assay, and also shows that myosin II is a downstream component of the LPA signaling pathway that leads to cell motility.

The present invention was also tested in vivo. Nude mice were inoculated with human melanoma cell line A2058 subcutaneously in the right flank and observed until the tumor was palpable (about 5-8 days). The mice were then treated with oral gavage of a bithionol-cyclodextrin suspension (A-D) or a cyclodextrin suspension alone (E-H). Panels A-D.—All 5 mice treated with bithionol lacked gross evidence of metastatic melanoma upon necropsy. Dissection of the tumor from the underlying fascia was easily accomplished for all animals (demonstrated in A&B). Microscopically, the tumors appeared as self-contained balls of tumor cells (C) with a pushing border. Higher powered examination of the tumor border reveals tumor encapsulated by benign fibroblasts and connective tissue (D). Panels E-H—3 of the 4 untreated mice showed gross and/or microscopic evidence of invasive melanoma. Two of the untreated mice developed large tumors in the right hindquarter (cyan arrow, E) that grossly invaded the right gluteous maximus and could not be dissected without amputation (E&F). Microscopic examination of the tumor revealed an invasive tumor which encompassed the muscle and bone of the right leg (G). Higher power examination of the tumor interface reveals tumor cells invading into and splaying apart normal muscle cells (H). Of the remaining untreated mice, one developed a modest sized tumor with microscopic evidence of invasion, and a one developed a tumor without gross or microscopic evidence of invasive tumor. The effects of these in vivo tests of bithionol suppression of melanoma invasion and metastasisare shown in FIG. 14.

In another in vivo experiment, murine mouse carcinoma cell line 66cl4 was injected into the left mammary fat pad of mice and allowed to grow for 48 hours. The mice were then orally dosed with alternate daily doses of bithionol-cyclodextrin (6 animals, 60/mg/kg) or cyclodextrin alone (6 animals) for 24 days. FIG. 15 shows the effect of ATX inhibition on breast cancer tumor growth. A. Scatterplot of the weights of tumor for both experimental groups. B. Average tumor weights of both experimental groups. The average tumor weight of the control animals was 0.297±0.116 g, and the average weight of the dosed animals was 0.141±0.107 g. the difference in weight was statistically significant (p=0.019). C. The weights of the animals in the two experimental groups did not differ significantly from each other over the course of the experiment.

These experiments validate ATX as a molecular target for metastatic melanoma, and demonstrate the feasibility of developing high affinity small molecule inhibitors of ATX capable of inhibiting its melanoma migration and growth and metastasis. These studies also point the way to in vivo studies, as one of the lead compounds (bithionol) has received FDA approval as a second line orally administered medication for the treatment of helmithic infection that can be safely dosed in humans at 25 mg kg⁻¹ body weight per day [43]. Detailed toxicology and pharmacokinetics on the drug are available, and oral doses of 50 mg/kg in 3 divided alternate daily doses for 5 days maintain serum levels in the range 140-550 μM in rabbits, dogs, and humans, or several times greater than those required to observe a 50% inhibition of melanoma cell migration in our in vitro assays. In the case of NSC 48300, while the exposure to arylarsonic acids may arouse concern, pharmaceutically more acceptable alternatives may be found utilizing elements within the same group of the periodic table (e.g. antimony), which are equally potent. These studies lay the groundwork for future in vivo experiments to test the extent to which ATX inhibitors will be useful in preventing melanoma spread and growth and metastasis.

In addition, in further experiments, assays were conducted and showed that ATX stimulates pericyte migration, which is a key process in the formation of mature and stable angiogenesis. The results of the cell migration assays evidences that ATX enhances angiogenesis, and inhibitors of ATX are agents for in inhibiting angiogenesis, treating cancer, inhibiting growth and metastasis and treating angiogenesis related disease states and conditions.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

### REFERENCES

1. Stracke M L, Krutzsch H C, Unsworth E J, et al. Identification, purification, and partial sequence analysis of autotaxin, a novel motility-stimulating protein. J Biol Chem 1992; 267:2524-2529.
2. Gijsbers R, Aoki J, Arai H, Bollen M. The hydrolysis of lysophospholipids and nucleotides by autotaxin (NPP2) involves a single catalytic site. FEBS Lett 2003; 538:60-64.
3. Lee H Y, Murata J, Clair T, et al. Cloning, chromosomal localization, and tissue expression of autotaxin from human teratocarcinoma cells. Biochem Biophys Res Commun 1996; 218:714-719.
4. Tokumura A, Majima E, Kariya Y, et al. Identification of human plasma lysophospholipase D, a lysophosphatidic acid-producing enzyme, as autotaxin, a multifunctional phosphodiesterase. J Biol Chem 2002; 277:39436-39442.
5. Umezu-Goto M, Kishi Y, Taira A, et al. Autotaxin has lysophospholipase D activity leading to tumor cell growth and motility by lysophosphatidic acid production. J Cell Biol 2002; 158:227-233.
6. Sakagami H, Aoki J, Natori Y, et al. Biochemical and molecular characterization of a novel choline-specific glycerophosphodiester phosphodiesterase belonging to the nucleotide pyrophosphatase/phosphodiesterase family. J Biol Chem 2005; 280:23084-23093.
7. Duan R D, Bergman T, Xu N, et al. Identification of human intestinal alkaline sphingomyelinase as a novel ecto-enzyme related to the nucleotide phosphodiesterase family. Biol Chem 2003; 278:38528-38536.
8. Koh E, Clair T, Woodhouse E C, Schiffmann E, Liotta L, Stracke M. Site-directed mutations in the tumor-associated cytokine, autotaxin, eliminate nucleotide phosphodiesterase, lysophospholipase D, and motogenic activities. Cancer Res 2003; 63:2042-2045.
9. Clair T, Aoki J, Koh E, et al. Autotaxin hydrolyzes sphingosylphosphorylcholine to produce the regulator of migration, sphingosine-1-phosphate. Cancer Res 2003; 63:5446-5453.
10. van Meeteren L A, Ruurs P, Stortelers C, er al. Autotaxin, a secreted lysophospholipase D, is essential for blood vessel formation during development. Mol Cell Biol 2006; 26:5015-5022.
11. Luquain C, Sciorra V A, Morris A J. Lysophosphatidic acid signaling: how a small lipid does big things. Trends Biochem Sci 2003; 28:377-383.
12. Nam S W, Clair T, Campo C K, Lee H Y, Liotta L A, Stracke M L. Autotaxin (ATX), a potent tumor motogen, augments invasive and metastatic potential of ras-transformed cells. Oncogene 2000; 19:241-247.
13. Nam S W, Clair T, Kim Y S, et al. Autotaxin (NPP-2), a metastasis-enhancing motogen, is an angiogenic factor. Cancer Res 2001; 61:6938-6944.
14. Tanaka M, Okudaira S, Kishi Y, et al. Autotaxin stabilizes blood vessels and is required for embryonic vasculature by producing lysophosphatidic acid. J Biol Chem 2006; 281:25822-25830.
15. Hoelzinger D B, Mariani L, Weis J, et al. Gene expression profile of glioblastoma multiforme invasive phenotype points to new therapeutic targets. Neoplasia 2005; 7:7-16.
16. Baumforth K R, Flavell J R, Reynolds G M, et al. Induction of autotaxin by the Epstein-Barr virus promotes the growth and survival of Hodgkin lymphoma cells. Blood 2005; 106:2138-2146.
17. Xie Y, Gibbs T C, Mukhin Y V, Meier K E. Role for 18:1 lysophosphatidic acid as an autocrine mediator in prostate cancer cells. J Biol Chem 2002; 277:32516-32526.
18. Mulvaney P T, Stracke M L, Nam S W, et al. Cyclocreatine inhibits stimulated motility in tumor cells possessing creatine kinase. Int J Cancer 1998; 78:46-52.
19. Quinones L G, Garcia-Castro I. Characterization of human melanoma cell lines according to their migratory properties in vitro. In Vitro Cell Dev Biol Anim 2004; 40:35-42.
20. Yang S Y, Lee J, Park C G, et al. Expression of autotaxin (NPP-2) is closely linked to invasiveness of breast cancer cells. Clin Exp Metastasis 2002; 19:603-608.
21. Kawagoe H, Stracke M L, Nakamura H, Sano K. Expression and transcriptional regulation of the PD-Ialpha/autotaxin gene in neuroblastoma. Cancer Res 1997; 57:2516-2521.
22. Kishi Y, Okudaira S, Kishi M, et al. Autotaxin is overexpressed in glioblastoma multiforme and contributes to cell motility of glioblastoma by converting lysophosphatidylcoholine to lysophosphatidic acid. J Biol Chem 2006; 25:17492-17500
23. Yang Y, Mou L, Liu N, Tsao M S. Autotaxin expression in non-small-cell lung cancer. Am J Respir Cell Mol Biol 1999; 21:216-222.
24. Kehlen A, Englert N, Seifert A, et al. Expression, regulation and function of autotaxin in thyroid carcinomas. Int J Cancer 2004; 109:833-838.
25. Mills G B, May C, McGill M, Roifman C M, Mellors A. A putative new growth factor in ascitic fluid from ovarian cancer patients: identification, characterization, and mechanism of action. Cancer Res 1988; 48:1066-1071.
26. Mills G B, May C, Hill M, Campbell S, Shaw P, Marks A. Ascitic fluid from human ovarian cancer patients contains growth factors necessary for intraperitoneal growth of human ovarian adenocarcinoma cells. J Clin Invest 1990; 86:851-855.
27. Xu Y, Gaudette D C, Boynton J D, et al. Characterization of an ovarian cancer activating factor in ascites from ovarian cancer patients. Clin Cancer Res 1995; 1:1223-1232.
28. Xiao Y J, Schwartz B, Washington M, et al. Electrospray ionization mass spectrometry analysis of lysophospholipids in human ascitic fluids: comparison of the lysophospholipid contents in malignant vs nonmalignant ascitic fluids. Anal Biochem 2001; 290:302-313.
29. Cui P, Tomsig J L, McCalmont W F, et al. Synthesis and biological evaluation of phosphonate derivatives as autotaxin (ATX) inhibitors. Bioorg Med Chem Lett 2007; 17:1634-1640.
30. Jiang G, Xu Y, Fujiwara Y, et al. alpha-Substituted Phosphonate Analogues of Lysophosphatidic Acid (LPA) Selectively Inhibit Production and Action of LPA. Chem Med Chem 2007; 2:679-690.
31. Baker D L, Fujiwara Y, Pigg K R, et al: Carba analogs of cyclic phosphatidic acid are selective inhibitors of autotaxin and cancer cell invasion and metastasis. J Biol Chem 2006; 281:22786-22793.
32. Liu X W, Sok D E, Yook H S, Sohn C B, Chung Y J, Kim M R. Inhibition of lysophospholipase D activity by unsaturated lysophosphatidic acids or seed extracts containing 1-linoleoyl and 1-oleoyl lysophosphatidic acid. J Agric Food Chem 2007; 55:8717-8722.
33. Prestwich G D, Gajewiak J, Zhang H, Xu X, Yang G, Serban M. Phosphatase-resistant analogues of lysophosphatidic acid: Agonists promote healing, antagonists and autotaxin inhibitors treat cancer. Biochim Biophys Acta 2008 Apr. 8 epub ahead of print.
34. O'Reilly D R, Miller L K, Lucklow V A. Bacculovirus Expression Vectors: A Laboratory Manual. Oxford University Press; 1994.
35. Zhang J H, Chung T D, Oldenburg K R. A Simple Statistical Parameter for Use in Evaluation and Validation of High Throughput Screening Assays. J Biomol Screen 1999; 4:67-73.
36. Ferguson C G, Bigman C S, Richardson R D, van Meeteren L A, Moolenaar W H, Prestwich G D. Fluorogenic phospholipid substrate to detect lysophospholipase D/autotaxin activity. Org Lett 2006; 8:2023-2026.
37. Birdsall B, King R W, Wheeler M R, et al. Correction for light absorption in fluorescence studies of protein-ligand interactions. Anal Biochem 1983; 132:353-361.
38. Nam S W, Clair T, Schiffmann E, Liotta L A, Stracke M L. A sensitive screening assay for secreted motility-stimulating factors. Cell Motil Cytoskeleton 2000; 46:279-284.
39. Fogg A G, Gray A, Burns D T. Stability constants of metal complexes of bithionol, fenticlor and hexachlorophene. Anal Chim Acta 1970; 51:265-270.
40. Duxbury M S, Ashley S W, Whang E E. Inhibition of pancreatic adenocarcinoma cellular invasiveness by blebbistatin: a novel myosin II inhibitor. Biochem Biophys Res Commun 2004; 313:992-997.
41. Lee H Y, Bae G U, Jung I D, et al. Autotaxin promotes motility via G protein-coupled phosphoinositide 3-kinase gamma in human melanoma cells. FEBS Lett 2002; 515:137-140.
42. Gelb M H, Jain M K, Hanel A M, Berg O G. Interfacial enzymology of glycerolipid hydrolases: lessons from secreted phospholipases A2. Annu Rev Biochem 1995; 64:653-688.
43. Bacq Y, Besnier J M, Duong T H, Pavie G, Metman E H, Choutet P. Successful treatment of acute fascioliasis with bithionol. Hepatology 1991; 14:1066-1069.

1. A method of inhibiting growth and/or metastasis of a cancer in a patient comprising administering to said patient an effective amount of an autotaxin inhibitor.

2. A method of treating cancer in a patient comprising administering to said patient an effective amount of an autotaxin inhibitor.

3. A method of inhibiting angiogenesis or treating an angiogenic disease related in a patient comprising administering to said patient and effective amount of an autotaxin inhibitor to said patient.

4. The method according to any of claims 1-3 wherein said compound is according to the chemical structure: 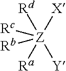
Where Z is a 5- or 6-membered ring containing up to four heteroatoms (O, S, N) or together with X′ or Y′, forms an optionally substituted fused ring system containing two or three rings, wherein said rings may be saturated or unsaturated, carbocyclic or heterocyclic (including aromatic or heteroaromatic);
X′ and Y′ are each independently H, optionally substituted heterocyclic, aryl or heteroaryl, wherein said heterocyclic, aryl or heteroaryl is optionally bonded to said Z group through a linker group L, halogen, an optionally substituted alkyl, OR′, where R′ is H, an optionally substituted C₁-C₆ alkyl (preferably C₁-C₃ alkyl), —C(O)—(C₁-C₆ alkyl),
—C(O)R″, where R″ is H, OH, an optionally substituted C₁-C₆ alkyl, O—(C₁-C₆ alkyl), NR^{Na}R^{Nb}, where R^{Na} is H or a C₁-C₆ alkyl and R^{Nb} is H, an optionally substituted C₁-C₃ alkyl or a C(O)R^{Nc} or C(O)OR^{Nc} group, where R^{Nc} is an optionally substituted C₁-C₁₂ hydrocarbyl group (including an aryl group), an optionally substituted saturated or unsaturated heterocyclic group (including a heteroaromatic group), a —AsO₃ group, a
Hg—O—R^{HG} group where R^{HG} is H or a C₁-C₃ alkyl group, a S(O)ₖR^{f} group, where R^{f} is H, an optionally substituted C₁-C₁₂ hydrocarbyl, heterocyclic or heteroaromatic group or a NR^{Nd}R^{Ne} group, where R^{Nd} is H or an optionally substituted C₁-C₆ hydrocarbyl group and R^{Ne} is H, or an optionally substituted C₁-C₁₂ hydrocarbyl group (preferably substituted with a S(O)ₖR^{fa} group, where R^{fa} is H, an optionally substituted C₁-C₁₂ hydrocarbyl, heterocyclic or heteroaromatic group, or a NR^{Nfa}R^{Nfe} group, where R^{Nfa} is H or an optionally substituted C₁-C₆ hydrocarbyl group and R^{Nfe} is H or an optionally substituted C₁-C₁₂ hydrocarbyl group;
L is a linker group of the general structure: 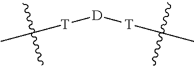
Where T and T′ are each independently a bond, —(CH₂)ᵢ—O, —(CH₂)ᵢ—S, —(CH₂)ᵢ—N—R, 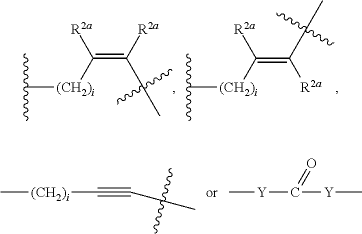
wherein said —(CH₂); group, if present in T or T′, is bonded to Z or X or Y;
R is H, or a C₁-C₃ alkyl group;
R^{2a} is H or a C₁-C₃ alkyl group;
Each Y is independently a bond, O, S or N—R;
Each i is independently 0, 1, 2 or 3;
k is 0, 1 or 2;
D is O, S, or N—H; 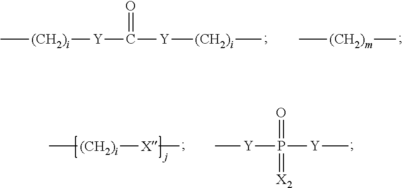
Where X₂ is O or is absent (along with the double bond);
i is the same as described above;
j is 1, 2, 3 or 4,
m is 1, 2, 3, 4, 5 or 6;
n is 1, 2 or 3; and
X″ is O, S or N—R;
R is H, or a C₁-C₃ alkyl group;
R^{a}, R^{b}, R^{c} and R^{d} are each independently absent (because the heteroatom is O or S and cannot accommodate a substituent) H, halogen (preferably F, Cl or Br), optionally substituted C₁-C₆ alkyl, OH, CN, NO₂, C(O)H, C(O)OH, O—(C₁-C₆ alkyl), C(O)(C₁-C₆
C(O)—O—(C₁-C₆ alkyl), —O—C(O)—(C₁-C₆ alkyl), a AsO₃ group or a Hg—O—R^{HG} group where R^{HG} is H or a C₁-C₃ alkyl group, with the proviso that no more than two substituents contain Hg or As;
or a pharmaceutically acceptable salt, solvate or polymorph thereof.

5. The method according to claim 4 wherein said compound is according to the chemical structure: 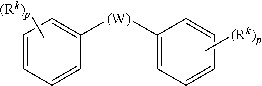
Where W is (CH₂)ᵢ, O, S or NR_{T};
i is 0, 1, 2 or 3;
R_{T} is H or C₁-C₃ alkyl (preferably H or CH₃);
Each R^{k} is independently OH, halogen (F, Cl, Br, I), C(O)H, C(O)OH, O—(C₁-C₆ alkyl), C(O)(C₁-C₆ alkyl), C(O)—O—(C₁-C₆ alkyl), —O—C(O)—(C₁-C₆ alkyl), a AsO₃ group or a Hg—O—R^{HG} group where R^{HG} is H or a C₁-C₃ alkyl group, with the proviso that no more than two substituents contain Hg or As
p is 1, 2, 3, 4 or 5, preferably
or a pharmaceutical salt solvate or polymorph thereof.

6. The method according to claim 4 wherein said compound is according to the chemical structure: 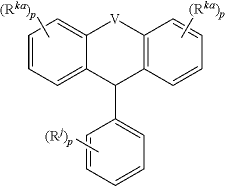
Where V is O, S or NR_{T};
R_{T} is H or C₁-C₃ alkyl (preferably H or CH₃);
Each R^{ka} is independently OH, CN, NO₂, halogen (F, Cl, Br, I), C(O)H, C(O)OH, O—(C₁-C₆ alkyl), C(O)(C₁-C₆ alkyl), C(O)—O—(C₁-C₆ alkyl), —O—C(O)—(C₁-C₆ alkyl), a AsO₃ group or a Hg—O—R^{HG} group where R^{HG} is H or a C₁-C₃ alkyl group, with the proviso that no more than two substituents R^{ka} contain Hg or As;
Each R^{j} is independently OH, halogen (F, Cl, Br, I), C(O)H, C(O)OH, O—(C₁-C₆ alkyl), C(O)(C₁-C₆ alkyl), C(O)—O—(C₁-C₆ alkyl), —O—C(O)—(C₁-C₆ alkyl), a AsO₃ group or a Hg—O—R^{HG} group where R^{HG} is H or a C₁-C₃ alkyl group, with the proviso that no more than two substituents contain Hg or As;
p is 1, 2, 3, 4 or 5, preferably 1-3;
or a pharmaceutical salt, solvate or polymorph thereof.

7. The method according to claim 4 wherein said compound is a saturated or unsaturated heterocyclic ring according to the chemical structure: 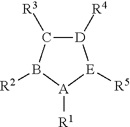
Where A, B, C, D or E are each a carbon, nitrogen, oxygen or sulphur atom (preferably carbon or nitrogen) with the proviso that at least two of A, B, C, D and E are carbon (preferably at least three of A, B, C, D and E are carbon and preferably A, C and E are carbon atoms and the other two atoms are nitrogen atoms);
R¹, R² and R³ are each independently absent (because the heteroatom cannot accommodate a substituent), H, halogen, an optionally substituted C₁-C₆ alkyl group, OH, O—(C₁-C₆ alkyl), O—C(O)—(C₁-C₆ alkyl), C(O)—(C₁-C₆ alkyl), C(O)—O—(C₁-C₆ alkyl), a AsO₃ group or a Hg—O—R^{HG} group where R^{HG} is H or a C₁-C₃ alkyl group, with the proviso that no more than two substituents of R¹, R² and R³ contain Hg or As or an optionally substituted 5- or 6 membered saturated or unsaturated carbocyclic or heterocyclic ring;
R⁴ and R⁵ are each independently absent (because the heteroatom cannot accommodate a substituent), H, halogen, optionally substituted C₁-C₆ alkyl or a NR^{g}R^{h}, where R^{g} is H, a C₁-C₃ group and R^{h} is H, a C₁-C₆ hydrocarbyl group or a C(O)—R^{h′} group where R^{h′} is an optionally substituted C₁-C₁₂ hydrocarbyl group or an optionally substituted heterocyclic group, or together R⁴ and R⁵ together form an optionally substituted five or six-membered saturated or unsaturated carbocyclic or heterocyclic group; or
a pharmaceutically acceptable salt, solvate or polymorph thereof.

8. The method according to claim 4 wherein said compound has the chemical structure: 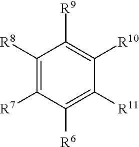
Where R⁶, R⁷, R⁸ and R⁹ are each independently selected from H, halogen, optionally substituted C₁-C₆ alkyl, OH, O—(C₁-C₆ alkyl), O—C(O)—(C₁-C₆ alkyl), C(O)—(C₁-C₆ alkyl), and C(O)—O—(C₁-C₆ alkyl), a AsO₃ group or a Hg—O—R^{HG} group where R^{HG} is H or a C₁-C₃ alkyl group or together R⁸ and R⁹ form an optionally substituted 5- or 6-membered saturated or unsaturated carbocyclic or heterocyclic ring (preferably optionally substituted aromatic or heteroaromatic);
R¹⁰ is a halogen, optionally substituted C₁-C₆ alkyl, OH, O—(C₁-C₆ alkyl), O—C(O)—(C₁-C₆ alkyl), C(O)—(C₁-C₆ alkyl), and C(O)—O—(C₁-C₆ alkyl), a AsO₃ group or a Hg—O—R^{HG} group where R^{HG} is H or a C₁-C₃ alkyl group 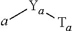
group, where Yₐ is S, an optionally substituted —(CH₂)_{q} group where q is 0, 1, 2, 3 or 4 (preferably 1) or an amine group which is optionally substituted with a single C₁-C₃ alkyl group and Tₐ is an optionally substituted aromatic or heteroaromatic group, or a S(O)ₖR^{f} group, where k is 0, 1 or 2 and R^{f} is H, an optionally substituted C₁-C₁₂ hydrocarbyl, heterocyclic or heteroaromatic group or a NR^{Nd}R^{Ne} group, where R^{Nd} is H or an optionally substituted C₁-C₆ hydrocarbyl group and R^{Ne} is H, or an optionally substituted C₁-C₁₂ hydrocarbyl group (preferably substituted with a S(O)ₖR^{fa} group, where R^{fa} is H, an optionally substituted C₁-C₁₂ hydrocarbyl, heterocyclic or heteroaromatic group, or a NR^{Nfa}R^{Nfe} group, where R^{Nfa} is H or an optionally substituted C₁-C₆ hydrocarbyl group and R^{Nfe} is H or an optionally substituted C₁-C₁₂ hydrocarbyl group, or together with R¹¹ and the aromatic ring to which they are attached, form an optionally substituted saturated or unsaturated carbocyclic or heterocyclic tricyclic ring system; and
R¹¹ is H, halogen, optionally substituted C₁-C₆ alkyl, OH, O—(C₁-C₆ alkyl), O—C(O)—(C₁-C₆ alkyl), C(O)—(C₁-C₆ alkyl), and C(O)—O—(C₁-C₆ alkyl), a AsO₃ group or a Hg—O—R^{HG} group where R^{HG} is H or a C₁-C₃ alkyl group an optionally substituted C₁-C₁₂ hydrocarbyl group, an optionally substituted heterocyclic group (preferably, an optionally substituted heteroaryl group) or together with R¹⁰ and the aromatic ring to which they are attached, form an optionally substituted saturated or unsaturated carbocyclic or heterocyclic tricyclic ring system; or
a pharmaceutically acceptable salt, solvate and polymorph thereof.

9. (canceled)

10. The method according to any of claims 1-3 wherein said compound is a compound as set forth in FIG. 2.

11. The method according to any of claims 1-3 wherein said compound is p-nitrophenol 5′ thymidine monophosphate (pNP-TMP), FS-3, NSC 10881, NSC 86629, NSC 13792, NSC 50016, NSC 78785 or mixtures thereof.

12. The method according to claim 1 or 2 wherein said cancer is stomach, colon, rectal, liver, pancreatic, lung, breast, cervix uteri, corpus uteri, ovary, prostate, testis, bladder, renal, thyroid, brain/CNS, head and neck, throat, Hodgkin's disease, non-Hodgkin's lymphoma, multiple myeloma, leukemia, melanoma, non-melanoma skin cancer, acute lymphocytic leukemia, acute myelogenous leukemia, Ewing's sarcoma, small cell lung cancer, choriocarcinoma, gliablastoma, rhabdomyosarcoma, Wilms' tumor, neuroblastoma, hairy cell leukemia, mouth/pharynx, esophagus, larynx, kidney cancer or lymphoma.

13. The method according to claim 1 or 2 wherein said cancer is brain cancer, melanoma, breast, prostate, ovarian, lung, stomach, colon or thyroid cancer.

14. The method according to claim 3 wherein said angiogenic disease is macular degeneration, diabetic retinopathy, psoriasis, venous ulcers, acne, rosacea, warts, eczema, hemangiomas, lymphangiogenesis, Sturge-Weber syndrome, neurofibromatosis, tuberous sclerosis, chronic inflammatory disease and arthritis.

15. The method according to claim 14 wherein said angiogenic disease is exudative (wet) macular degeneration or diabetic retinopathy.

16. A compound according to the chemical structure: 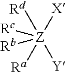
Where Z is a 5- or 6-membered ring containing up to four heteroatoms (O, S, N) or together with X′ or Y′, forms an optionally substituted fused ring system containing two or three rings, wherein said rings may be saturated or unsaturated, carbocyclic or heterocyclic (including aromatic or heteroaromatic);
X′ and Y′ are each independently H, optionally substituted heterocyclic, aryl or heteroaryl, wherein said heterocyclic, aryl or heteroaryl is optionally bonded to said Z group through a linker group L, halogen, an optionally substituted alkyl, OR′, where R′ is H, an optionally substituted C₁-C₆ alkyl (preferably C₁-C₃ alkyl), —C(O)—(C₁-C₆ alkyl), —C(O)R″, where R″ is H, OH, an optionally substituted C₁-C₆ alkyl, O—(C₁-C₆ alkyl), NR^{Na}R^{Nb}, where R^{Na} is H or a C₁-C₆ alkyl and R^{Nb} is H, an optionally substituted C₁-C₃ alkyl or a C(O)R^{Nc} or C(O)OR^{Nc} group, where R^{Nc} is an optionally substituted C₁-C₁₂ hydrocarbyl group (including an aryl group), an optionally substituted saturated or unsaturated heterocyclic group (including a heteroaromatic group), a —AsO₃ group, a
Hg—O—R^{HG} group where R^{HG} is H or a C₁-C₃ alkyl group, a S(O)ₖR^{f} group, where R^{f} is H, an optionally substituted C₁-C₁₂ hydrocarbyl, heterocyclic or heteroaromatic group or a NR^{Nd}R^{Ne} group, where R^{Nd} is H or an optionally substituted C₁-C₆ hydrocarbyl group and R^{Ne} is H, or an optionally substituted C₁-C₁₂ hydrocarbyl group (preferably substituted with a S(O)ₖR^{fa} group, where R^{fa} is H, an optionally substituted C₁-C₁₂ hydrocarbyl, heterocyclic or heteroaromatic group, or a NR^{Nfa}R^{Nfe} group, where R^{Nfa} is H or an optionally substituted C₁-C₆ hydrocarbyl group and R^{Nfe} is H or an optionally substituted C₁-C₁₂ hydrocarbyl group;
L is a linker group of the general structure: 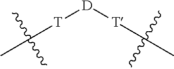
Where T and T′ are each independently a bond, —(CH₂)ᵢ—O, —(CH₂)ᵢ—S, —(CH₂)ᵢ—N—R, 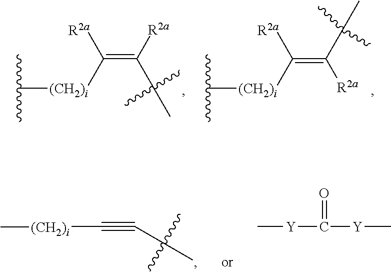
wherein said —(CH₂), group, if present in T or T′, is bonded to Z or X or Y;
R is H, or a C₁-C₃ alkyl group;
R^{2a} is H or a C₁-C₃ alkyl group;
Each Y is independently a bond, O, S or N—R;
Each i is independently 0, 1, 2 or 3;
k is 0, 1 or 2;
D is O, S, or N—H; 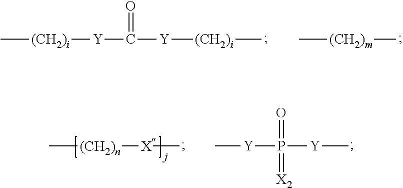
Where X₂ is O or is absent (along with the double bond);
i is the same as described above;
j is 1, 2, 3 or 4,
m is 1, 2, 3, 4, 5 or 6;
n is 1, 2 or 3; and
X″ is O, S or N—R;
R is H, or a C₁-C₃ alkyl group;
R^{a}, R^{b}, R^{c} and R^{d} are each independently absent (because the heteroatom is O or S and cannot accommodate a substituent) H, halogen (preferably F, Cl or Br), optionally substituted C₁-C₆ alkyl, OH, CN, NO₂, C(O)H, C(O)OH, O—(C₁-C₆ alkyl), C(O)(C₁-C₆ alkyl), C(O)—O—(C₁-C₆ alkyl), —O—C(O)—(C₁-C₆ alkyl), a AsO₃ group or a Hg—O—R^{HG} group where R^{HG} is H or a C₁-C₃ alkyl group, with the proviso that no more than two substituents contain Hg or As;
or a pharmaceutically acceptable salt, solvate or polymorph thereof.

17. The compound according to claim 16 according to the chemical structure: 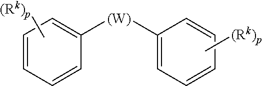
Where W is (CH₂)ᵢ, O, S or NR_{T},
i is 0, 1, 2 or 3;
R_{T} is H or C₁-C₃ alkyl (preferably H or CH₃);
Each R^{k} is independently OH, halogen (F, Cl, Br, I), C(O)H, C(O)OH, O—(C₁-C₆ alkyl), C(O)(C₁-C₆ alkyl), C(O)—O—(C₁-C₆ alkyl), —O—C(O)—(C₁-C₆ alkyl), a AsO₃ group or a Hg—O—R^{HG} group where R^{HG} is H or a C₁-C₃ alkyl group, with the proviso that no more than two substituents contain Hg or As
p is 1, 2, 3, 4 or 5, preferably
or a pharmaceutical salt solvate or polymorph thereof.

18. The compound according to claim 16 according to the chemical structure: 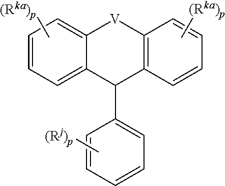
Where V is O, S or NR_{T},
R_{T} is H or C₁-C₃ alkyl (preferably H or CH₃);
Each R^{ka} is independently OH, CN, NO₂, halogen (F, Cl, Br, I), C(O)H, C(O)OH, O—(C₁-C₆ alkyl), C(O)(C₁-C₆ alkyl), C(O)—O—(C₁-C₆ alkyl), —O—C(O)—(C₁-C₆ alkyl), a AsO₃ group or a Hg—O—R^{HG} group where R^{HG} is H or a C₁-C₃ alkyl group, with the proviso that no more than two substituents R^{ka} contain Hg or As;
Each R^{j} is independently OH, halogen (F, Cl, Br, I), C(O)H, C(O)OH, O—(C₁-C₆ alkyl), C(O)(C₁-C₆ alkyl), C(O)—O—(C₁-C₆ alkyl), —O—C(O)—(C₁-C₆ alkyl), a AsO₃ group or a Hg—O—R^{HG} group where R^{HG} is H or a C₁-C₃ alkyl group, with the proviso that no more than two substituents contain Hg or As;
p is 1, 2, 3, 4 or 5, preferably 1-3;
or a pharmaceutical salt, solvate or polymorph thereof.

19. The compound according to claim 16 according to the chemical structure: 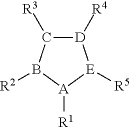
Where A, B, C, D or E are each a carbon, nitrogen, oxygen or sulphur atom (preferably carbon or nitrogen) with the proviso that at least two of A, B, C, D and E are carbon (preferably at least three of A, B, C, D and E are carbon and preferably A, C and E are carbon atoms and the other two atoms are nitrogen atoms);
R¹, R² and R³ are each independently absent (because the heteroatom cannot accommodate a substituent), H, halogen, an optionally substituted C₁-C₆ alkyl group, OH, O—(C₁-C₆ alkyl), O—C(O)—(C₁-C₆ alkyl), C(O)—(C₁-C₆ alkyl), C(O)—O—(C₁-C₆ alkyl), a AsO₃ group or a Hg—O—R^{HG} group where R^{HG} is H or a C₁-C₃ alkyl group, with the proviso that no more than two substituents of R¹, R² and R³ contain Hg or As or an optionally substituted 5- or 6 membered saturated or unsaturated carbocyclic or heterocyclic ring;
R⁴ and R⁵ are each independently absent (because the heteroatom cannot accommodate a substituent), H, halogen, optionally substituted C₁-C₆ alkyl or a NR^{g}R^{h}, where R^{g} is H, a C₁-C₃ group and R^{h} is H, a C₁-C₆ hydrocarbyl group or a C(O)—R^{h′} group where R^{h′} is an optionally substituted C₁-C₁₂ hydrocarbyl group or an optionally substituted heterocyclic group, or together R⁴ and R⁵ together form an optionally substituted five or six-membered saturated or unsaturated carbocyclic or heterocyclic group; or
a pharmaceutically acceptable salt, solvate or polymorph thereof.

20. The compound according to claim 16 according to the chemical structure: 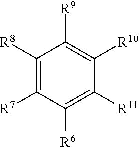
Where R⁶, R⁷, R⁸ and R⁹ are each independently selected from H, halogen, optionally substituted C₁-C₆ alkyl, OH, O—(C₁-C₆ alkyl), O—C(O)—(C₁-C₆ alkyl), C(O)—(C₁-C₆ alkyl), and C(O)—O—(C₁-C₆ alkyl), a AsO₃ group or a Hg—O—R^{HG} group where R^{HG} is H or a C₁-C₃ alkyl group or together R⁸ and R⁹ form an optionally substituted 5- or 6-membered saturated or unsaturated carbocyclic or heterocyclic ring (preferably optionally substituted aromatic or heteroaromatic);
R¹⁰ is a halogen, optionally substituted C₁-C₆ alkyl, OH, O—(C₁-C₆ alkyl), O—C(O)—(C₁-C₆ alkyl), C(O)—(C₁-C₆ alkyl), and C(O)—O—(C₁-C₆ alkyl), a AsO₃ group or a Hg—O—R^{HG} group where R^{HG} is H or a C₁-C₃ alkyl group 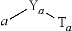
group, where Yₐ is S, an optionally substituted —(CH₂)_{q} group where q is 0, 1, 2, 3 or 4 (preferably 1) or an amine group which is optionally substituted with a single C₁-C₃ alkyl group and Tₐ is an optionally substituted aromatic or heteroaromatic group, or a S(O)ₖR^{f} group, where k is 0, 1 or 2 and R^{f} is H, an optionally substituted C₁-C₁₂ hydrocarbyl, heterocyclic or heteroaromatic group or a NR^{Nd}R^{Ne} group, where R^{Nd} is H or an optionally substituted C₁-C₆ hydrocarbyl group and R^{Ne} is H, or an optionally substituted C₁-C₁₂ hydrocarbyl group (preferably substituted with a S(O)ₖR^{fa} group, where R^{fa} is H, an optionally substituted C₁-C₁₂ hydrocarbyl, heterocyclic or heteroaromatic group, or a NR^{Nfa}R^{Nfe} group, where R^{Nfa} is H or an optionally substituted C₁-C₆ hydrocarbyl group and R^{Nfe} is H or an optionally substituted C₁-C₁₂ hydrocarbyl group, or together with R¹¹ and the aromatic ring to which they are attached, form an optionally substituted saturated or unsaturated carbocyclic or heterocyclic tricyclic ring system; and
R¹¹ is H, halogen, optionally substituted C₁-C₆ alkyl, OH, O—(C₁-C₆ alkyl), O—C(O)—(C₁-C₆ alkyl), C(O)—(C₁-C₆ alkyl), and C(O)—O—(C₁-C₆ alkyl), a AsO₃ group or a Hg—O—R^{HG} group where R^{HG} is H or a C₁-C₃ alkyl group an optionally substituted C₁-C₁₂ hydrocarbyl group, an optionally substituted heterocyclic group (preferably, an optionally substituted heteroaryl group) or together with R¹⁰ and the aromatic ring to which they are attached, form an optionally substituted saturated or unsaturated carbocyclic or heterocyclic tricyclic ring system; or
a pharmaceutically acceptable salt, solvate and polymorph thereof.

21. (canceled)

22. A pharmaceutical composition comprising an effective amount of a compound according to any of claims 16-20, optionally in combination with a pharmaceutically acceptable carrier, additive or excipient.

23. The composition according to claim 22 further comprising an additional anticancer agent or other bioactive agent in an effective amount.

24. The composition according to claim 23 wherein said additional anticancer agent is selected from the group consisting of antimetabolites, inhibitors of topoisomerase I and II, alkylating agents, microtubule inhibitors, tyrosine kinase inhibitors, EGF kinase inhibitors and ABL kinase inhibitors or mixtures thereof.

25. The composition according to claim 23 wherein said additional anticancer agent is selected from the group consisting of Aldesleukin; Alemtuzumab; alitretinoin; allopurinol; altretamine; amifostine; anastrozole; arsenic trioxide; Asparaginase; BCG Live; bexarotene capsules; bexarotene gel; bleomycin; busulfan intravenous; busulfan oral; calusterone; capecitabine; carboplatin; carmustine; carmustine with Polifeprosan 20 Implant; celecoxib; chlorambucil; cisplatin; cladribine; cyclophosphamide; cytarabine; cytarabine liposomal; dacarbazine; dactinomycin; actinomycin D; Darbepoetin alfa; daunorubicin liposomal; daunorubicin, daunomycin; Denileukin diftitox, dexrazoxane; docetaxel; doxorubicin; doxorubicin liposomal; Dromostanolone propionate; Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemtuzumab ozogamicin; gleevec (imatinib); goserelin acetate; hydroxyurea; Ibritumomab Tiuxetan; idarubicin; ifosfamide; imatinib mesylate; Interferon alfa-2a; Interferon alfa-2b; irinotecan; letrozole; leucovorin; levamisole; lomustine (CCNU); meclorethamine (nitrogen mustard); megestrol acetate; melphalan (L-PAM); mercaptopurine (6-MP); mesna; methotrexate; methoxsalen; mitomycin C; mitotane; mitoxantrone; nandrolone phenpropionate; Nofetumomab; LOddC; Oprelvekin; oxaliplatin; paclitaxel; pamidronate; pegademase; Pegaspargase; Pegfilgrastim; pentostatin; pipobroman; plicamycin; mithramycin; porfimer sodium; procarbazine; quinacrine; Rasburicase; Rituximab; Sargramostim; streptozocin; surafenib; talbuvidine (LDT); talc; tamoxifen; tarceva (erlotinib); temozolomide; teniposide (VM-26); testolactone; thioguanine (6-TG); thiotepa; topotecan; toremifene; Tositumomab; Trastuzumab; tretinoin (ATRA); Uracil Mustard; valrubicin; valtorcitabine (monoval LDC); vinblastine; vinorelbine; zoledronate; and mixtures thereof.

26. The composition according to claim 23 wherein said bioactive agent is an antiviral agent or an analgesic agent.

27. (canceled)

28. The composition according to claim 23 wherein said bioactive agent is the anti-VEGF agent bevacizumab, ranibizumab, sunitinib, sorafenib, axitinib, pazopanib or a mixture thereof.

29-36. (canceled)
